SEQUENCE LISTING (90 sequences)

SEQ ID NO: 1 - DNA, 94 bp, synthetic construct
  source 1..94: mol_type = other DNA
gtttccgcgaggtgcggattgaaaatggtctgctgctgctgaacggcaagccgttgctgattcgaggcgttaaccgttacgactttaaccataa

SEQ ID NO: 2 - DNA, 22 bp, synthetic construct
  source 1..22: mol_type = other DNA
ttatggttaaagtcgtaaccgt

SEQ ID NO: 3 - DNA, 61 bp, synthetic construct
  source 1..61: mol_type = other DNA
tccgcatactgaatcagagatacttgcgctcgttcgctacgactttaaccataagttggac

SEQ ID NO: 4 - DNA, 52 bp, synthetic construct
  source 1..52: mol_type = other DNA
gttcaacttatggttaaagtcgtattcgccagccaggacagaaatgcctcga

SEQ ID NO: 5 - DNA, 45 bp, synthetic construct
  source 1..45: mol_type = other DNA
gttcgcattatccgaaccatccgctgtggtacacgctgtgcgacc

SEQ ID NO: 6 - protein, 318 aa, Nostoc sp.
MMLSFFPILYPDELLYSGLARYHIRSGNRSFKQTDLELFGYSSQQVCKVTLTNNLNHLVNNLSLLSQQTINNLLQKHTLYPFYAILLMPQEAWLLKSSMSKKINESILEVAKMTNGSGGNSTKYLKFCHSCVGEDTQKYGEPYWHRLHQIPGVIVCPIHRIPLNNSLVPIETKEIHYHAPSDDNCPLNTGTTIYNDATLQKLLVFANDIEWLINNNFTFQGLSWLRSQYKTYLTNKNFITVFSKDKFIFHEQEFYNAVLAYYGQDFLEAINPKRIKNPDKYLSNCLLACDLNPVIDRVMHILIIKFLANSIEDFFKAQ

SEQ ID NO: 7 - DNA, 39 bp, Nostoc sp.
  source 1..39: mol_type = other DNA
atgcggaagattgtgatcaatttaactcccgcagattta

SEQ ID NO: 8 - DNA, 36 bp, Nostoc sp.
  source 1..36: mol_type = other DNA
cgaaatattgtgattaatttcactcctgctgattta

SEQ ID NO: 9 - protein, 315 aa, Nostoc linckia
MLSFFPTLYPDELLYSALARYHIRSGNKSFRQTDIELFGFHSQQLSKVTLTNNLNYLVNNLPFYSRKRVDHLLCNHTLYPFYASFLTQQEIFLLGDSIKKKFHGSVFEIAKLSLKSTGNEKKFLKFCPVCLEEEIQQYGEPYWHRSHQIPGIYVCLNHNSFLHDSTVMIETKGIHYHAASSENCLRSDSQFSDSYQTLTQLLILAKDIEWLISSNFCFQGLSWLRNQYQSYLIKREFLTVLPGNKLKLHETELCQSIFEMYSQDFLSIVNINFIRNPAKYLSHCLLACDVNPVIDRITHILMIKFLANSLEWFFI

SEQ ID NO: 10 - DNA, 39 bp, Nostoc linckia
  source 1..39: mol_type = other DNA
atgcggaagattgtaattaacttgactccggctgatttg

SEQ ID NO: 11 - DNA, 36 bp, Nostoc linckia
  source 1..36: mol_type = other DNA
cggaagattgtgattaacttgacttcggctgattta

SEQ ID NO: 12 - protein, 315 aa, Phormidium tenue
MLTLPKPYVDELLYSILVRYYIRSGYRKVKEAQVKLFDTLPQQPWDILLPSNLKRLTRKLWTKANYTPDYFIQGHTLYPFYAQFLIPVETELLRQVMVQQGRASVPTIAKIPLNVEKACHSYLKFCPQCFEQESDELGEAYWHRTHQIPGIVLCPDHEVPLLNSTVCLNSKALHYIAADSDTCPINNNVPSYTDLTKHRMTAYTESLERLIDRQIPFRGLAWLRKRYHHYAAQKGFLKFDTATNFTFDETKFFEELCDFYGEEFLDNILPVSFQSSKHQFIQCLLACDLEQTIDRVRHILLINFLSDSLQDFFAY

SEQ ID NO: 13 - DNA, 49 bp, Phormidium tenue
  source 1..49: mol_type = other DNA
atggcgcaggttgtttggctgcagtggtggttaatcccaattcgattga

SEQ ID NO: 14 - DNA, 46 bp, Phormidium tenue
  source 1..46: mol_type = other DNA
gcgcaagtggtttggcttcactgatggctcacctcggtgctttaag

SEQ ID NO: 15 - protein, 315 aa, Phormidium sp.
MVNFLPHPYPDEHFYSLLTRCHMRSADKKLRKTLKGLLGYSSKKLFRQDLPDGLSNLMMSLPPASPHFVEDLIQNHTLYPFYKSFLTPSEAWLLKHRMIKATNESFISLAKLSPDGLDSNRKFLQFCPACLEEEEARYGEAYWHRMHQAPGVFVCSNHKVPLQDSLIPLHNIDREYVPANTYNCPNNRSKNRYSEVALQTLLTLYDDIEWLMYSAPSFKGLKWLRKRYQTFLTQQDYVSTLPKSKSDFNSQTLFEDITNFYGLEVLDLIKPDKVANMKVYLECCLLACDIDQVIDRITHLLLIKFLSGSLEHFFN

SEQ ID NO: 16 - DNA, 37 bp, Phormidium sp.
  source 1..37: mol_type = other DNA
atgtggaggagaaagcacccactggcaagctctatgt

SEQ ID NO: 17 - DNA, 34 bp, Phormidium sp.
  source 1..34: mol_type = other DNA
tggatgaaaaagcaccccctggcaagctgtatgt

SEQ ID NO: 18 - protein, 318 aa, Nostoc sp.
MLNGFPRIYPDELLYSVIARYHIRNAYKSFHQSDMELFGYASQQIYRVVLPCNLNHLVREIHLHLFYELNINDLIYHHTLYPFYASFLPPQEAWLLKNYMEQKANVSLSEILKCPRNNKEEAKTFLKFCLYCIEEDTQKYGEPYWHRFHQVPGVIVCPIHRIALNNSLVSIETKEIHYHAPSDDNCPLNTSTTIYNDATLQKLLVFANNIEWLINNNFTFKGLSWLRSHYKTYLTNKNFITVFSKDKFIFHEQEFYNAVLTYYGQEFLEAINPKIIKNPEKYFSNCLLACDVNPVIDRIIHILIIKFLANSIEDFFKA

SEQ ID NO: 19 - DNA, 38 bp, Nostoc sp.
  source 1..38: mol_type = other DNA
atgcgaaaaattgtgatcaatttaactccggcagattt

SEQ ID NO: 20 - DNA, 35 bp, Nostoc sp.
  source 1..35: mol_type = other DNA
cggaaaattgtgattaagttcactcctgctgattt

SEQ ID NO: 21 - DNA, 106 bp, Phormidium sp.
  source 1..106: mol_type = other DNA
atgtggaggagaaagcacccactggcaagctctatgtaacggtgccactccttcaagcaacgggactccatccagggcagctggagttttggaaaacggttgaaca

SEQ ID NO: 22 - DNA, 38 bp, Phormidium sp.
  source 1..38: mol_type = other DNA
aattgttcaaccgtttttcaaaactccagcagcgcgtg

SEQ ID NO: 23 - protein, 30 aa, synthetic construct
IFQNDNSKCQSRENNSPPTLGVNETLKQYR

SEQ ID NO: 24 - protein, 12 aa, synthetic construct
TFQGYGHPCQSR

SEQ ID NO: 25 - protein, 22 aa, synthetic construct
TFQGYRNLWQHSAKTSERTPVI

SEQ ID NO: 26 - protein, 22 aa, synthetic construct
TFQGYNNGCLSKATALLPTSDI

SEQ ID NO: 27 - protein, 30 aa, synthetic construct
TLHDYNKYCQGEEDDAPKAHEVSEILALCE

SEQ ID NO: 28 - protein, 30 aa, synthetic construct
TLHDYNKYCQGEEEDSPKAHKVEEILRLCR

SEQ ID NO: 29 - protein, 30 aa, synthetic construct
TLHDYNKYCQGEEEDAPKTHEVSEILGLCH

SEQ ID NO: 30 - protein, 30 aa, synthetic construct
TLHDYNKYCQGEEDDPPKAYEVDSILALCH

SEQ ID NO: 31 - protein, 30 aa, synthetic construct
TLHDYNKYCNGQGEETPKNSEVTEIVNRCR

SEQ ID NO: 32 - protein, 30 aa, synthetic construct
TLHDYNKYCSGQGEETPQAYEVPTILELCQ

SEQ ID NO: 33 - protein, 30 aa, synthetic construct; identical to SEQ ID NO: 32

SEQ ID NO: 34 - protein, 30 aa, synthetic construct
TLHDYNKYCLGHGEESPKVSNINEIINICQ

SEQ ID NO: 35 - protein, 30 aa, synthetic construct
TLHDYDKHCRSQVKKPPHPSDVPAILEVCQ

SEQ ID NO: 36 - protein, 30 aa, synthetic construct
TLHDYNKYCNGQGEETPKNWETEEIIDLCR

SEQ ID NO: 37 - protein, 30 aa, synthetic construct
TLHDYNKYCNGQGEETPKNWEVEKILNLCR

SEQ ID NO: 38 - protein, 30 aa, synthetic construct
TLHDYNKYCNGQGEETPNNWDVEQIINLCR

SEQ ID NO: 39 - protein, 30 aa, synthetic construct
TLHDYNKYRNGQGEETPKNSEVTEILNLCR

SEQ ID NO: 40 - protein, 30 aa, synthetic construct
TLHDYNKYCNGQGEETPKNWEVEEIINVCR

SEQ ID NO: 41 - protein, 30 aa, synthetic construct
TLHDYNKYCNGQGEETPKNSQVAEILNICR

SEQ ID NO: 42 - protein, 30 aa, synthetic construct
TLHDYNKYCQGEEKDSPKAYEVDSILALCQ

SEQ ID NO: 43 - protein, 30 aa, synthetic construct
TLHDYNKYYLGCGEKSPSAADVAEIINICR

SEQ ID NO: 44 - protein, 30 aa, synthetic construct; identical to SEQ ID NO: 43

SEQ ID NO: 45 - protein, 30 aa, synthetic construct
TLHDYNKYCNGQGEETPKNWEVEEIINLCR

SEQ ID NO: 46 - protein, 30 aa, synthetic construct
TLHDYNKYCNGQGEETPKNWQVEEILNVCR

SEQ ID NO: 47 - protein, 30 aa, synthetic construct
TLHDYNKYCNGQGEETPRNYEVDEIINLCR

SEQ ID NO: 48 - protein, 30 aa, synthetic construct
TLHDYDKHCRSQGIQPPGSDDIPAILKVCE

SEQ ID NO: 49 - protein, 30 aa, synthetic construct
TLHDYNKYCNGQGEETPKNWQVEEIINVCR

SEQ ID NO: 50 - protein, 30 aa, synthetic construct; identical to SEQ ID NO: 47

SEQ ID NO: 51 - protein, 30 aa, synthetic construct
TLHDYNKYCHAQGEETPKHWEVENIITLCH

SEQ ID NO: 52 - protein, 30 aa, synthetic construct
TLHDYNKYCNGQGEETPKNWQVEEIIDLCR

SEQ ID NO: 53 - protein, 30 aa, synthetic construct
TLHDYNKYCNGQGEETPNNWEVDGIINLCR

SEQ ID NO: 54 - protein, 30 aa, synthetic construct
TLHDYNKYCNGQGEETPKNWEVEEILNVCR

SEQ ID NO: 55 - protein, 30 aa, synthetic construct; identical to SEQ ID NO: 54

SEQ ID NO: 56 - protein, 31 aa, synthetic construct
TLHDYNKYCDAQGEDDPPKAYEVAEILKLCE

SEQ ID NO: 57 - protein, 30 aa, synthetic construct
TLHDYNKYCNGQGEESPKHWEVEEIINICQ

SEQ ID NO: 58 - protein, 30 aa, synthetic construct
TLHDYDKHCRSQVIQPPSSDNIPKILKICE

SEQ ID NO: 59 - protein, 32 aa, synthetic construct
TLHDYNKYVQGKGEEQPPPKAHEIEEIINLCQ

SEQ ID NO: 60 - protein, 32 aa, synthetic construct
TLHDYNKYVRGKGEEQPPPKAHEVEEIINLCQ

SEQ ID NO: 61 - protein, 30 aa, synthetic construct
TLHDYNKYCTGEEEDPPKAHEVEDILNLCR

SEQ ID NO: 62 - protein, 32 aa, synthetic construct
TLHDYNKAVQGQKEETAPPKASEIPQILQVCE

SEQ ID NO: 63 - protein, 32 aa, synthetic construct
TLHDYNKYVRGQGEEQPPPKAHEIDAIINLCR

SEQ ID NO: 64 - protein, 32 aa, synthetic construct; identical to SEQ ID NO: 63

SEQ ID NO: 65 - protein, 30 aa, synthetic construct
TLHDYNKYCIGGGEESPHASDVEAILIICQ

SEQ ID NO: 66 - protein, 32 aa, synthetic construct
TLHDYNKYVRGQGEEQPPPKAHEITAIINLCQ

SEQ ID NO: 67 - protein, 32 aa, synthetic construct
TLHDYNKAIQGQTEASTSPKAHEIRQILQVCE

SEQ ID NO: 68 - protein, 32 aa, synthetic construct
TLHDYNKVIQGQTEASTSPKAHEIRQILQVCE

SEQ ID NO: 69 - protein, 32 aa, synthetic construct; identical to SEQ ID NO: 68

SEQ ID NO: 70 - protein, 32 aa, synthetic construct
TLHDYNKIIQGQTEASTSPKAHEIRQILQVCE

SEQ ID NO: 71 - protein, 32 aa, synthetic construct; identical to SEQ ID NO: 68

SEQ ID NO: 72 - protein, 32 aa, synthetic construct; identical to SEQ ID NO: 70

SEQ ID NO: 73 - protein, 7 aa, Myxacorys californica
LLVHILN

SEQ ID NO: 74 - protein, 7 aa, Synechocystis sp.
LAAHILN

SEQ ID NO: 75 - protein, 7 aa, Myxacorys californica
LIFQNDN

SEQ ID NO: 76 - protein, 7 aa, Myxacorys californica
LIFQNDN

SEQ ID NO: 77 - protein, 7 aa, Synechocystis sp.
ITLHDYD

SEQ ID NO: 78 - protein, 9 aa, Myxacorys californica
FGAIAAQLT

SEQ ID NO: 79 - protein, 9 aa, Synechocystis sp.
FGDVAVHLS

SEQ ID NO: 80 - DNA, 51 bp, synthetic construct
  source 1..51: mol_type = other DNA
cttttttcccaaggaaatattgttatgaccgaaaaattgaaactgactaaa

SEQ ID NO: 81 - protein, 17 aa, synthetic construct
LFSQGNIVMTEKLKLTK

SEQ ID NO: 82 - protein, 17 aa, synthetic construct
VWAAGDSNMEQQLELTQ

SEQ ID NO: 83 - DNA, 51 bp, synthetic construct
  source 1..51: mol_type = other DNA
gtatgggcagcaggagattcaaacatggaacagcaattggagctaactcag

SEQ ID NO: 84 - RNA, 19 nt, synthetic construct
  source 1..19: mol_type = other RNA
acatgaggatcacccatgt

SEQ ID NO: 85 - DNA, 25 bp, synthetic construct
  source 1..25: mol_type = other DNA
ccgcgctgtactggaggctgaagtt

SEQ ID NO: 86 - DNA, 38 bp, synthetic construct
  source 1..38: mol_type = other DNA
ttggtctcttcagctcctcatgtaaaagtgtcttcaaa

SEQ ID NO: 87 - DNA, 18 bp, synthetic construct
  source 1..18: mol_type = other DNA
cagcgaccagatgatcac

SEQ ID NO: 88 - DNA, 37 bp, synthetic construct
  source 1..37: mol_type = other DNA
ttggtctctccaattaccagcaccatgatctttataa

SEQ ID NO: 89 - DNA, 58 bp, synthetic construct
  source 1..58: mol_type = other DNA
gttgctcttcaagagttgcccggcgctctccggctgcccggcttccattcaggtcgag

SEQ ID NO: 90 - DNA, 58 bp, synthetic construct
  source 1..58: mol_type = other DNA
gttgctcttcatctggctcacagtacgcgtagtgcnnnntgcagaatccctgcttcgt

(19) United States
(12) Patent Application Publication
(10) Pub. No.: US 2025/0243513 A1
(43) Pub. Date: Jul. 31, 2025

(54) TYPE I-D CRISPR-GUIDED TRANSPOSON WITH ENHANCED GENOME EDITING

(71) Applicant: Cornell University, Ithaca, NY (US)

(72) Inventors: Joseph E. PETERS, Ithaca, NY (US); Shan-Chi HSIEH, Ithaca, NY (US)

(21) Appl. No.: 18/869,576

(22) PCT Filed: Jun. 5, 2023
(86) PCT No.: PCT/US2023/067942

§ 371 Date: Nov. 26, 2024

Related U.S. Application Data
(60) Provisional application No. 63/348,895, filed on Jun. 3, 2022.

(51) Int. Cl. C12N 15/90; C12N 9/22; C12N 15/113

(52) U.S. Cl. CPC C12N 15/902; C12N 9/22; C12N 15/113; C12N 2310/12; C12N 2310/20; C12N 2800/90

(57) ABSTRACT

Provided are type I-D CRISPR-associated transposon (CAST) systems. The systems can be used with modified guide RNAs that are self-processing, and can be adapted to include binding sites for non-CAST proteins or polynucleotides. The systems may exclude a Cas6 protein. Methods of using the CAST systems for modifying DNA in heterologous hosts are also included.

### CROSS-REFERENCE TO RELATED APPLICATION

This application is based on and claims priority to U.S. Patent Application No. 63/348,895, filed on Jun. 3, 2022, the entire disclosure of which is incorporated herein by reference.

### STATEMENT REGARDING FEDERALLY SPONSORED RESEARCH

This invention was made with government support under grant numbers R01GM129118 and R21AI148941 awarded by the National Institutes of Health. The government has certain rights in the invention.

### SEQUENCE LISTING

The instant application contains a Sequence Listing which has been submitted in .xml format and is hereby incorporated by reference in its entirety. Said .xml copy was created on Jun. 5, 2023, is named “018617_01418_ST26.xml,” and is 82,467 bytes in size.

### BACKGROUND OF THE DISCLOSURE

This disclosure relates to mobile elements known as CRISPR-associated transposons (CASTs). All of the CAST systems that have been previously characterized are Tn7-like systems with a core set of 3-4 transposition genes that coopted CRISPR-Cas domain proteins from independent subtypes. There is an ongoing and unmet need for new or improved CRISPR systems. The present disclosure is pertinent to this need.

### SUMMARY OF THE DISCLOSURE

This disclosure provides type I-D CRISPR-Cas systems for use in guide RNA-directed DNA modification. The described systems can use variable length guide RNAs which can be designed for auto-maturation via ribozymes allowing independence from the steps normally required from Cas6. In more detail, the present disclosure provides systems that include recombinantly produced or isolated type I-D CRISPR-associated transposon (CAST) proteins. The systems may exclude a Cas6 protein. The CAST proteins include a TnsC protein, a TnsD protein, a TniQ protein, a fusion protein comprising TnsA and TnsB proteins, a Cas5 protein, Cas7 protein, and a Cas10 protein. The systems include a guide-RNA comprising a sequence targeted to a target within a DNA substrate. In embodiments, at least one of the CAST proteins comprises an amino acid sequence that is at least 50% identical to a protein that is encoded by Myxacorys californica WJT36-NPBG1. In non-limiting examples, the guide RNA is modified such that it is lengthened compared to guide RNAs in other CRISPR systems, and/or the guide RNA can be modified to comprise protein binding sites, or polynucleotide binding sites, or a combination thereof. The CAST proteins can be modified to include additional amino acids, such as a nuclear localization signal. Expression vectors encoding CAST proteins and a optionally encoding a guide RNA are included in the disclosure. Ribonucleoproteins comprising a described system are included. A described system may also include a DNA cargo for insertion into DNA substrate in a guide RNA directed manner. The disclosure includes introducing into cells a described system such that a DNA substrate is modified by using the guide RNA to direct the system to a selected target sequence.

### BRIEF DESCRIPTION OF THE FIGURES

For a fuller understanding of the nature and objects of the disclosure, reference should be made to the following detailed description taken in conjunction with the accompanying figures.

FIG. 1, panels A-D, provide diagram representations of a bioinformatics analysis to reveal a family of CAST disclosed herein.

FIGS. 2A-C provide diagram and graphical representations of in vivo transposition assay of the type I-D CRISPR-guided pathway and TnsD mediated tRNA-targeting with McCAST in E. coli. Sequences on FIG. 2B, top line nucleotide sequence, before double squiggle:

(SEQ ID NO: 1)
GTTTCCGCGAGGTGCGGATTGAAAATGGTCTGCTGCTGCTGAACGGCAAG
CCGTTGCTGATTCGAGGCGTTAACCGTTACGACTTTAACCATAA

after double squiggle: TTATGGTTAAAGTCGTAACCGT (SEQ ID NO:2).

Sequences on FIG. 2C, top line nucleotide sequence, before and after the double squiggle

Before:

(SEQ ID NO: 3)
TCCGCATACTGAATCAGAGATACTTGCGCTCGTTCGCTACGACTTTAACC
ATAAGTTGGAC.

After:

(SEQ ID NO: 4)
GTTCAACTTATGGTTAAAGTCGTATTCGCCAGCCAGGACAGAAATGCCTC
GA.

FIGS. 3A-3C provide diagram and graphical representations of the PAM preference of McCAST disclosed herein. FIG. 3A nucleotide sequence:

(SEQ ID NO: 5)
GTTCGCATTATCCGAACCATCCGCTGTGGTACACGCTGTGCGACC

FIGS. 4A-4B provide graphical representations of the impact of extended spacers on McCAST transposition and the resulting insertion distributions.

FIGS. 5A-5E provide diagram and graphical representations of examining the requirement of Cas6d for RNA-guided transportation.

FIGS. 6A-6C provide diagram and graphical representations of the characteristics of McCAST disclosed herein.

FIG. 7A-7B provide diagram representations of the diversity and evolutionary flexibility of Tn7-like transposons with TnsAB fusion in cyanobacteria.

FIGS. 8A-8C provide diagram representations of the convergent evolution observed in type I-B1 CASTs. The amino acid sequences of FIGS. 8A and 8B are TniQ sequences.

FIG. 8A panel 1:

MBG1266647.1 transposase [Nostoc sp. WHI]
(SEQ ID NO: 6)
MMLSFFPILYPDELLYSGLARYHIRSGNRSFKQTDLELFGYSSQQVCKVT
LINNLNHLVNNLSLLSQQTINNLLQKHTLYPFYAILLMPQEAWLLKSSMS
KKINESILEVAKMTNGSGGNSTKYLKFCHSCVGEDTQKYGEPYWHRLHQI
PGVIVCPIHRIPLNNSLVPIETKEIHYHAPSDDNCPLNTGTTIYNDATLQ
KLLVFANDIEWLINNNFTFQGLSWLRSQYKTYLTNKNFITVFSKDKFIFH
EQEFYNAVLAYYGQDFLEAINPKRIKNPDKYLSNCLLACDLNPVIDRVMH
ILIIKFLANSIEDFFKAQ
First:
(SEQ ID NO: 7)
5′-ATGCGGAAGATTGTGATCAATTTAACTCCCGCAGATTTA-3′
Second:
(SEQ ID NO: 8)
5′-CGAAATATTGTGATTAATTTCACTCCTGCTGATTTA-3′

FIG. 8A panel 2:

MBD2211882.1 TniQ family protein [Nostoc linckia
FACHB-104]
(SEQ ID NO: 9)
MLSFFPTLYPDELLYSALARYHIRSGNKSFRQTDIELFGFHSQQLSKVTL
TNNLNYLVNNLPFYSRKRVDHLLCNHTLYPFYASFLTQQEIFLLGDSIKK
KFHGSVFEIAKLSLKSTGNEKKFLKFCPVCLEEEIQQYGEPYWHRSHQIP
GIYVCLNHNSFLHDSTVMIETKGIHYHAASSENCLRSDSQFSDSYQTLTQ
LLILAKDIEWLISSNFCFQGLSWLRNQYQSYLIKREFLTVLPGNKLKLHE
TELCQSIFEMYSQDFLSIVNINFIRNPAKYLSHCLLACDVNPVIDRITHI
LMIKFLANSLEWFFI
First:
(SEQ ID NO: 10)
5′-ATGCGGAAGATTGTAATTAACTTGACTCCGGCTGATTTG-3′
Second:
(SEQ ID NO: 11)
5′-CGGAAGATTGTGATTAACTTGACTTCGGCTGATTTA-3′

FIG. 8A panel 3:

MBD3885833.1 TniQ family protein [Phormidium tenue
FACHB-886]
(SEQ ID NO: 12)
MLTLPKPYVDELLYSILVRYYIRSGYRKVKEAQVKLFDTLPQQPWDILLP
SNLKRLTRKLWTKANYTPDYFIQGHTLYPFYAQFLIPVETELLRQVMVQQ
GRASVPTIAKIPLNVEKACHSYLKFCPQCFEQESDELGEAYWHRTHQIPG
IVLCPDHEVPLLNSTVCLNSKALHYIAADSDTCPINNNVPSYTDLTKHRM
TAYTESLERLIDRQIPFRGLAWLRKRYHHYAAQKGFLKFDTATNFTFDET
KFFEELCDFYGEEFLDNILPVSFQSSKHQFIQCLLACDLEQTIDRVRHIL
LINFLSDSLQDFFAY
First:
(SEQ ID NO: 13)
5′-ATGGCGCAGGTTGTTTGGCTGCAGTGGTGGTTAATCCCAATTCGATT
GA-3′
Second:
(SEQ ID NO: 14)
5′-GCGCAAGTGGTTTGGCTTCACTGATGGCTCACCTCGGTGCTTTAAG-
3′

FIG. 8A panel 4:

MBD2077006.1 TniQ family protein [Phormidium sp.
FACHB-592]
(SEQ ID NO: 15)
MVNFLPHPYPDEHFYSLLTRCHMRSADKKLRKTLKGLLGYSSKKLFRQDL
PDGLSNLMMSLPPASPHFVEDLIQNHTLYPFYKSFLTPSEAWLLKHRMIK
ATNESFISLAKLSPDGLDSNRKFLQFCPACLEEEEARYGEAYWHRMHQAP
GVFVCSNHKVPLQDSLIPLHNIDREYVPANTYNCPNNRSKNRYSEVALQT
LLTLYDDIEWLMYSAPSFKGLKWLRKRYQTFLTQQDYVSTLPKSKSDFNS
QTLFEDITNFYGLEVLDLIKPDKVANMKVYLECCLLACDIDQVIDRITHL
LLIKFLSGSLEHFFN
First:
(SEQ ID NO: 16)
5′-ATGTGGAGGAGAAAGCACCCACTGGCAAGCTCTATGT-3′
Second:
(SEQ ID NO: 17)
5′-TGGATGAAAAAGCACCCCCTGGCAAGCTGTATGT-3′

FIG. 8A panel *:

WP_094343310.1 TnsD family Tn7-like transposition
protein [Nostoc sp. ‘Peltigera membranacea
cyanobiont’ 232]
(SEQ ID NO: 18)
MLNGFPRIYPDELLYSVIARYHIRNAYKSFHQSDMELFGYASQQIYRVVL
PCNLNHLVREIHLHLFYELNINDLIYHHTLYPFYASFLPPQEAWLLKNYM
EQKANVSLSEILKCPRNNKEEAKTFLKFCLYCIEEDTQKYGEPYWHRFHQ
VPGVIVCPIHRIALNNSLVSIETKEIHYHAPSDDNCPLNTSTTIYNDATL
QKLLVFANNIEWLINNNFTFKGLSWLRSHYKTYLTNKNFITVFSKDKFIF
HEQEFYNAVLTYYGQEFLEAINPKIIKNPEKYFSNCLLACDVNPVIDRII
HILIIKFLANSIEDFFKA
First:
(SEQ ID NO: 19)
5′-ATGCGAAAAATTGTGATCAATTTAACTCCGGCAGATTT-3′
Second:
(SEQ ID NO: 20)
5′-CGGAAAATTGTGATTAAGTTCACTCCTGCTGATTT-3′

FIG. 8B, nucleotide sequence before and after the / /

Before:
(SEQ ID NO: 21)
5′-
ATGTGGAGGAGAAAGCACCCACTGGCAAGCTCTATGTAACGGTGCCACTC
CTTCAAGCAACGGGACTCCATCCAGGGCAGCTGGAGTTTTGGAAAACGGT
TGAACA-3′
After:
(SEQ ID NO: 22)
5′-AATTGTTCAACCGTTTTTCAAAACTCCAGCAGCGCGTG-3′

FIGS. 9A-9C provide diagram and table representations to demonstrate that transposon-associated type I-D CRISPR systems show features common to CAST systems. The FIG. 9B alignment sequences are:

| | | cov | pid | 201 | . . . . : | 250 |
|---|---|---|---|---|---|---|
| 1 | MBW4418978.1 | 100.0% | 100.0% | | IFQNDNSKCQSR------------ENN--SP-PTLGVNETL-----KQYR | |
| (SEQ ID NO: 23) | | | | | | |
| 2 | WP_224344603.1 | 96.0% | 69.6% | | TFQGYGHPCQSR-------------------------------------- | |
| (SEQ ID NO: 24) | | | | | | |
| 3 | WP_190646788.1 | 99.2% | 64.8% | | TFQGYRNLWQHS------------AKT--SE-RTPVI------------- | |
| (SEQ ID NO: 25) | | | | | | |
| 4 | MBD1866148.1 | 96.9% | 64.4% | | TFQGYNNGCLSK------------ATA--LL-PTSDI------------- | |
| (SEQ ID NO: 26) | | | | | | |
| 5 | MBD1847458.1 | 98.2% | 56.0% | | TLHDYNKYCQGE------------EDD--AP-KAHEVSEIL-----ALCE | |
| (SEQ ID NO: 27) | | | | | | |
| 6 | WP_194024837.1 | 98.9% | 52.9% | | TLHDYNKYCQGE------------EED--SP-KAHKVEEIL-----RLCR | |
| (SEQ ID NO: 28) | | | | | | |
| 7 | WP_080810414.1 | 98.9% | 52.2% | | TLHDYNKYCQGE------------EED--AP-KTHEVSEIL-----GLCH | |
| (SEQ ID NO: 29) | | | | | | |
| 8 | WP_215607749.1 | 98.9% | 49.9% | | TLHDYNKYCQGE------------EDD--PP-KAYEVDSIL-----ALCH | |
| (SEQ ID NO: 30) | | | | | | |
| 9 | WP_053457730.1 | 96.6% | 49.9% | | TLHDYNKYCNGQ------------GEE--TP-KNSEVTEIV-----NRCR | |
| (SEQ ID NO: 31) | | | | | | |
| 10 | WP_190434436.1 | 97.5% | 49.6% | | TLHDYNKYCSGQ------------GEE--TP-QAYEVPTIL-----ELCQ | |
| (SEQ ID NO: 32) | | | | | | |
| 11 | MBD1835388.1 | 97.6% | 49.4% | | TLHDYNKYCSGQ------------GEE--TP-QAYEVPTIL-----ELCQ | |
| (SEQ ID NO: 33) | | | | | | |
| 12 | WP_012593877.1 | 97.4% | 50.2% | | TLHDYNKYCLGH------------GEE--SP-KVSNINEII-----NICQ | |
| (SEQ ID NO: 34) | | | | | | |
| 13 | MBW4569448.1 | 97.4% | 50.1% | | TLHDYDKHCRSQ------------VKK--PP-HPSDVPAIL-----EVCQ | |
| (SEQ ID NO: 35) | | | | | | |
| 14 | MBR8833507.1 | 97.0% | 49.8% | | TLHDYNKYCNGQ------------GEE--TP-KNWETEEII-----DLCR | |
| (SEQ ID NO: 36) | | | | | | |
| 15 | WP_015127543.1 | 96.6% | 50.2% | | TLHDYNKYCNGQ------------GEE--TP-KNWEVEKIL-----NLCR | |
| (SEQ ID NO: 37) | | | | | | |
| 16 | WP_190469883.1 | 96.6% | 49.6% | | TLHDYNKYCNGQ------------GEE--TP-NNWDVEQII-----NLCR | |
| (SEQ ID NO: 38) | | | | | | |
| 17 | WP_190958006.1 | 96.6% | 49.2% | | TLHDYNKYRNGQ------------GEE--TP-KNSEVTEIL-----NLCR | |
| (SEQ ID NO: 39) | | | | | | |
| 18 | WP_035152549.1 | 97.2% | 49.4% | | TLHDYNKYCNGQ------------GEE--TP-KNWEVEEII-----NVCR | |
| (SEQ ID NO: 40) | | | | | | |
| 19 | BAY29769.1 | 96.6% | 49.1% | | TLHDYNKYCNGQ------------GEE--TP-KNSQVAEIL-----NICR | |
| (SEQ ID NO: 41) | | | | | | |
| 20 | NEZ54669.1 | 98.9% | 48.6% | | TLHDYNKYCQGE------------EKD--SP-KAYEVDSIL-----ALCQ | |
| (SEQ ID NO: 42) | | | | | | |
| 21 | ELR97243.1 | 96.8% | 49.0% | | TLHDYNKYYLGC------------GEK--SP-SAADVAEII-----NICR | |
| (SEQ ID NO: 43) | | | | | | |
| 22 | WP_034937246.1 | 96.6% | 49.8% | | TLHDYNKYYLGC------------GEK--SP-SAADVAEII-----NICR | |
| (SEQ ID NO: 44) | | | | | | |
| 23 | MBW4666114.1 | 96.6% | 48.4% | | TLHDYNKYCNGQ------------GEE--TP-KNWEVEEII-----NLCR | |
| (SEQ ID NO: 45) | | | | | | |
| 24 | WP_206268314.1 | 96.6% | 48.5% | | TLHDYNKYCNGQ------------GEE--TP-KNWQVEEIL-----NVCR | |
| (SEQ ID NO: 46) | | | | | | |
| 25 | WP_099071831.1 | 96.6% | 48.9% | | TLHDYNKYCNGQ------------GEE--TP-RNYEVDEII-----NLCR | |
| (SEQ ID NO: 47) | | | | | | |
| 26 | WP_088240873.1 | 98.1% | 48.3% | | TLHDYDKHCRSQ------------GIQ--PP-GSDDIPAIL-----KVCE | |
| (SEQ ID NO: 48) | | | | | | |
| 27 | WP_155752058.1 | 96.6% | 49.0% | | TLHDYNKYCNGQ------------GEE--TP-KNWQVEEII-----NVCR | |
| (SEQ ID NO: 49) | | | | | | |
| 28 | MBL1203067.1 | 96.6% | 48.6% | | TLHDYNKYCNGQ------------GEE--TP-RNYEVDEII-----NLCR | |
| (SEQ ID NO: 50) | | | | | | |
| 29 | WP_200989354.1 | 96.6% | 49.2% | | TLHDYNKYCHAQ------------GEE--TP-KHWEVENII-----TLCH | |
| (SEQ ID NO: 51) | | | | | | |
| 30 | WP_017750002.1 | 96.6% | 48.9% | | TLHDYNKYCNGQ------------GEE--TP-KNWQVEEII-----DLCR | |
| (SEQ ID NO: 52) | | | | | | |
| 31 | WP_190693493.1 | 96.6% | 48.8% | | TLHDYNKYCNGQ------------GEE--TP-NNWEVDGII-----NLCR | |
| (SEQ ID NO: 53) | | | | | | |
| 32 | MBH8564955.1 | 96.6% | 49.1% | | TLHDYNKYCNGQ------------GEE--TP-KNWEVEEIL-----NVCR | |
| (SEQ ID NO: 54) | | | | | | |
| 33 | WP_225896517. 1 | 96.6% | 49.0% | | TLHDYNKYCNGQ------------GEE--TP-KNWEVEEIL-----NVCR | |
| (SEQ ID NO: 55) | | | | | | |
| 34 | WP_046279385.1 | 97.8% | 48.1% | | TLHDYNKYCDAQ------------GED--DPPKAYEVAEIL-----KLCE | |
| (SEQ ID NO: 56) | | | | | | |
| 35 | MBF2066226.1 | 95.9% | 48.4% | | TLHDYNKYCNGQ------------GEE--SP-KHWEVEEII-----NICQ | |
| (SEQ ID NO: 57) | | | | | | |
| 36 | WP_036267899.1 | 98.2% | 46.5% | | TLHDYDKHCRSQ------------VIQ--PP-SSDNIPKIL-----KICE | |
| (SEQ ID NO: 58) | | | | | | |
| 37 | WP_096680032.1 | 97.6% | 47.0% | | TLHDYNKYVQGK------------GEEQPPP-KAHEIEEII-----NLCQ | |
| (SEQ ID NO: 59) | | | | | | |
| 38 | WP_190594918.1 | 97.7% | 47.5% | | TLHDYNKYVRGK------------GEEQPPP-KAHEVEEII-----NLCQ | |
| (SEQ ID NO: 60) | | | | | | |
| 39 | MBE9041410.1 | 96.2% | 47.9% | | TLHDYNKYCTGE------------EED--PP-KAHEVEDIL-----NLCR | |
| (SEQ ID NO: 61) | | | | | | |
| 40 | WP_190471364.1 | 97.8% | 47.5% | | TLHDYNKAVQGQ------------KEETAPP-KASEIPQIL-----QVCE | |
| (SEQ ID NO: 62) | | | | | | |
| 41 | WP_103136873.1 | 97.7% | 47.2% | | TLHDYNKYVRGQ------------GEEQPPP-KAHEIDAII-----NLCR | |
| (SEQ ID NO: 63) | | | | | | |
| 42 | WP_236141231.1 | 97.7% | 47.1% | | TLHDYNKYVRGQ------------GEEQPPP-KAHEIDAII-----NLCR | |
| (SEQ ID NO: 64) | | | | | | |
| 43 | WP_013334249.1 | 97.0% | 48.9% | | TLHDYNKYCIGG------------GEE--SP-HASDVEAIL-----IICQ | |
| (SEQ ID NO: 65) | | | | | | |
| 44 | WP_152592234.1 | 97.9% | 46.5% | | TLHDYNKYVRGQ------------GEEQPPP-KAHEITAII-----NLCQ | |
| (SEQ ID NO: 66) | | | | | | |
| 45 | WP_083617784.1 | 97.7% | 47.4% | | TLHDYNKAIQGQ------------TEASTSP-KAHEIRQIL-----QVCE | |
| (SEQ ID NO: 67) | | | | | | |
| 46 | WP_043938760.1 | 97.3% | 47.5% | | TLHDYNKVIQGQ------------TEASTSP-KAHEIRQIL-----QVCE | |
| (SEQ ID NO: 68) | | | | | | |
| 47 | WP_043937175.1 | 97.3% | 47.6% | | TLHDYNKVIQGQ------------TEASTSP-KAHEIRQIL-----QVCE | |
| (SEQ ID NO: 69) | | | | | | |
| 48 | WP_227398151.1 | 97.5% | 47.6% | | TLHDYNKIIQGQ------------TEASTSP-KAHEIRQIL-----QVCE | |
| (SEQ ID NO: 70) | | | | | | |
| 49 | WP_141293796.1 | 97.3% | 47.6% | | TLHDYNKVIQGQ------------TEASTSP-KAHEIRQIL-----QVCE | |
| (SEQ ID NO: 71) | | | | | | |
| 50 | WP_227381095.1 | 97.5% | 47.6% | | TLHDYNKIIQGQ------------TEASTSP-KAHEIRQIL-----QVCE | |
| (SEQ ID NO: 72) | | | | | | |
| 51 | WP_002791883.1 | 96.6% | 19.9% | | LVHDFEKFSYDRFPSMSERYIQIQRDFIQDPFKNQDPRKLSREEHREILQ | |
| (SEQ ID NO: 73) | | | | | | |

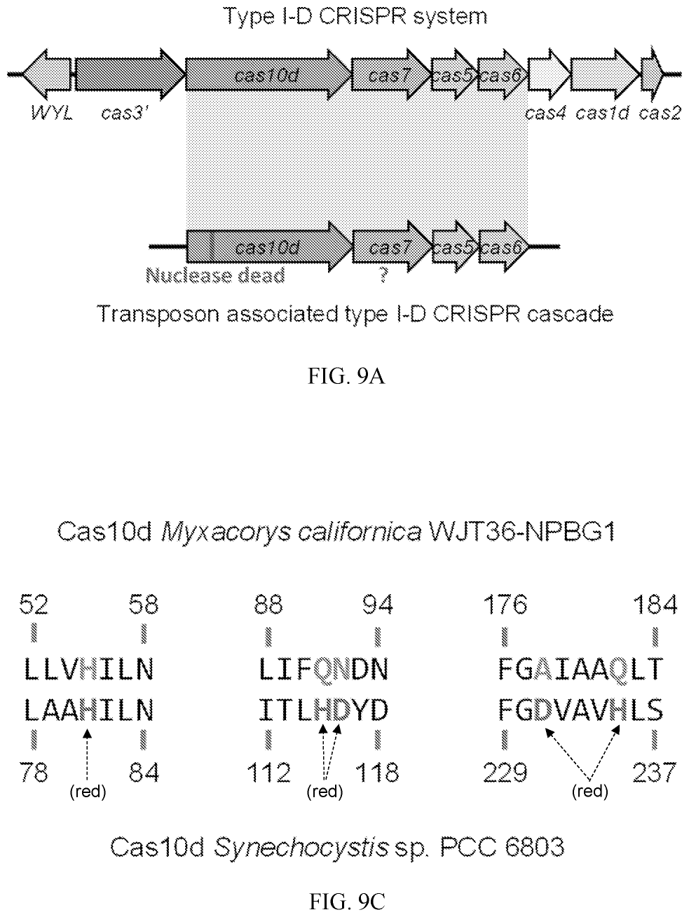

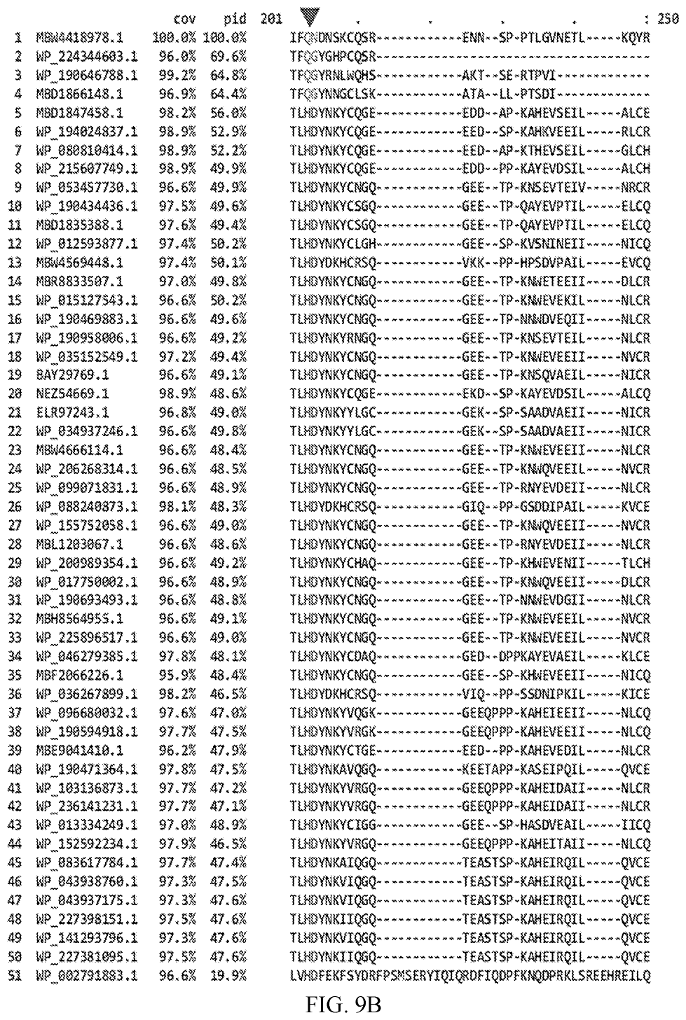

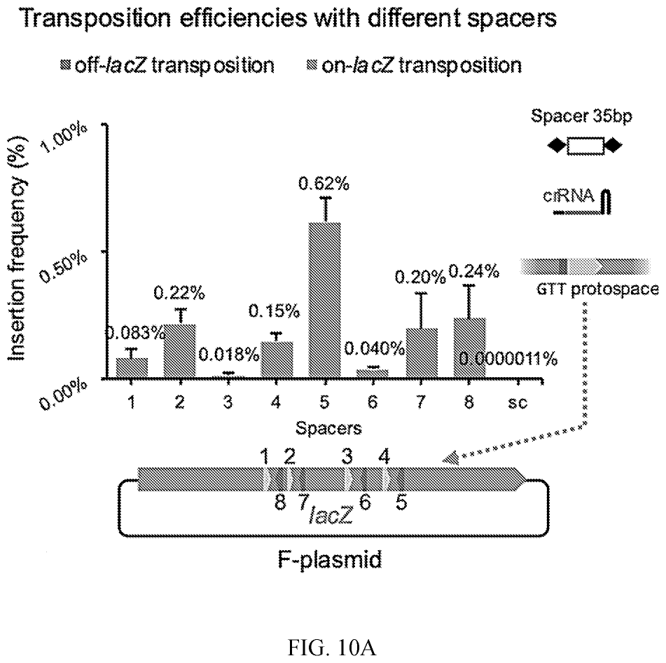

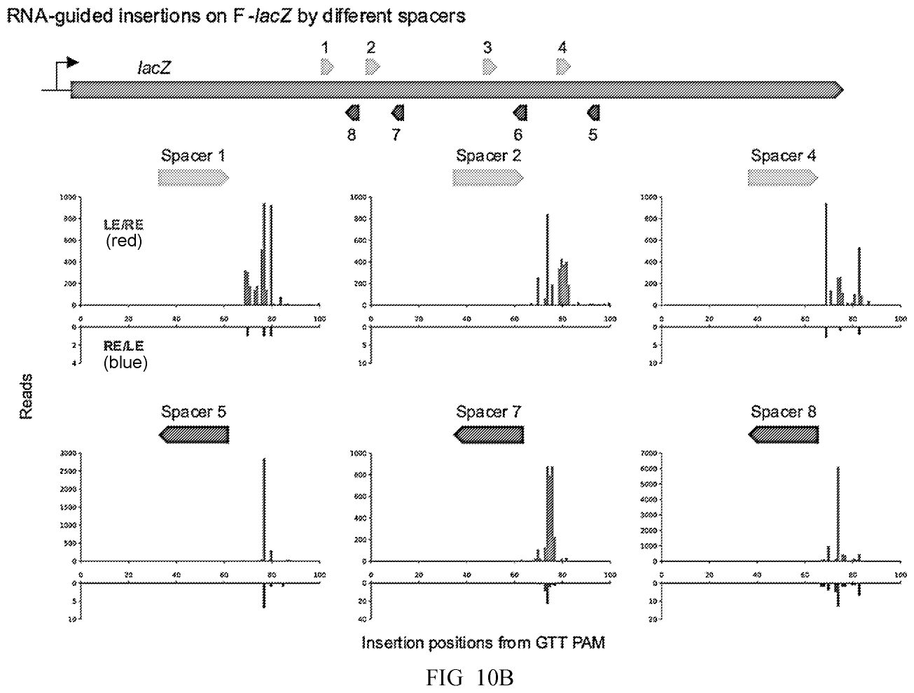

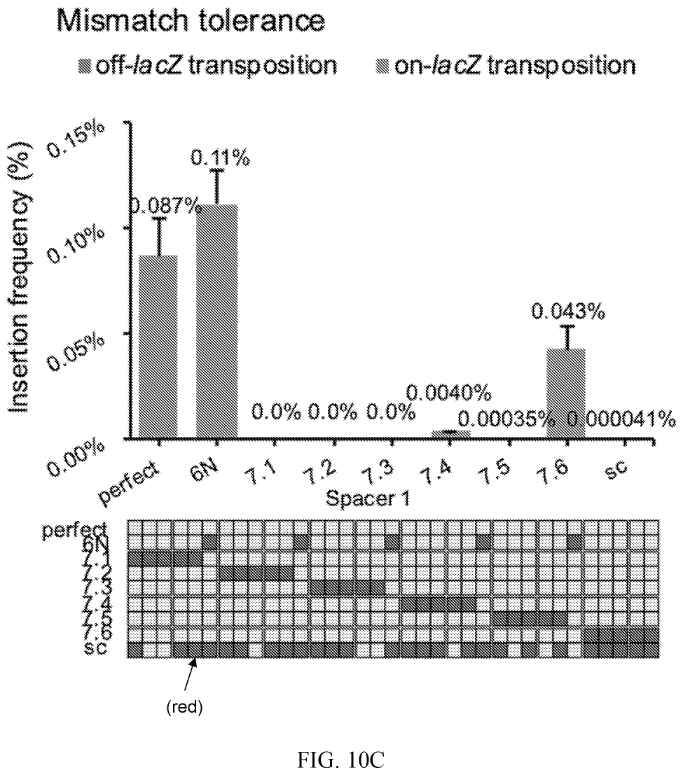

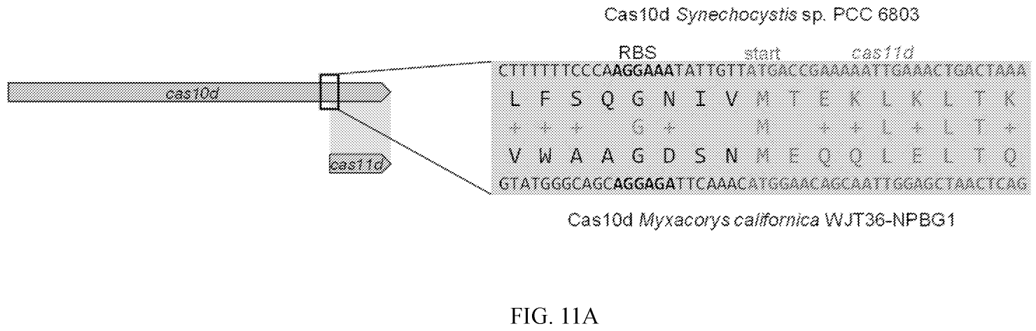

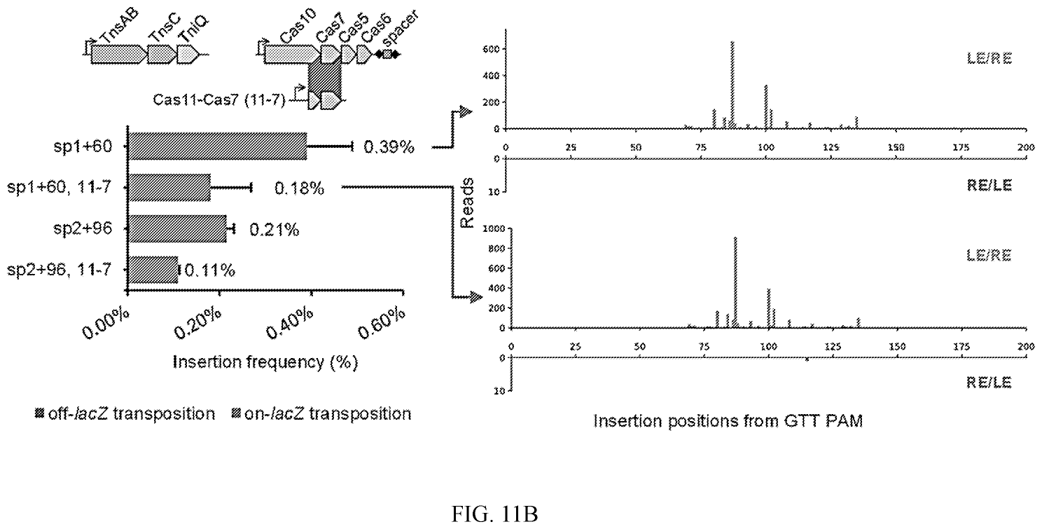

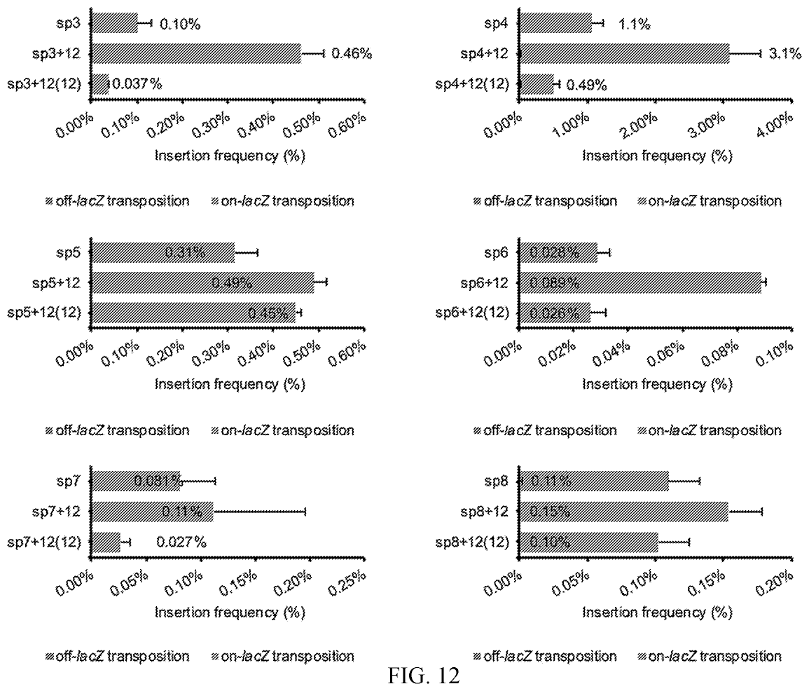

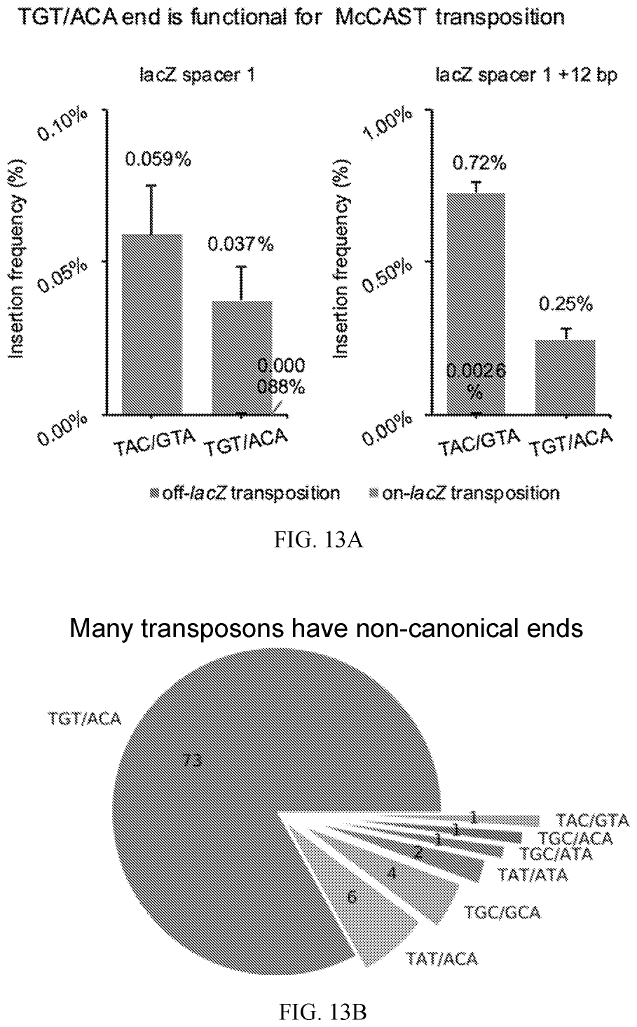

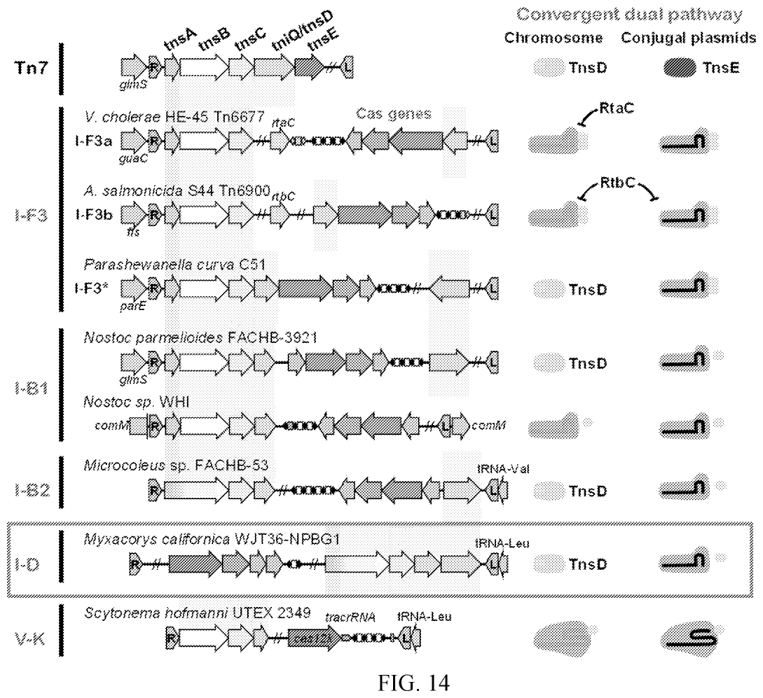

Sequences on FIG. 9C—Cas10d Myxacorys califorinica top line, Cas10d Synechocystis sp. PCC 6808 bottom line, each block of 7 amino acids separated:

Block 1:
Cas10d Myxacorys californica WJT36-NPBG1 (52-58)
N′-LLVHILN-C′ (SEQ ID NO:74)
Cas10d Synechocystis sp. PCC 6803 (78-84)
N′-LAAHILN-C′ (SEQ ID NO:75)

Block 2:
Cas10d Myxacorys californica WJT36-NPBG1 (88-94)
N′-LIFQNDN-C′ (SEQ ID NO:76)
50 Cas10d Synechocystis sp. PCC 6803 (112-118)
N′-ITLHDYD-C′ (SEQ ID NO:77)

Block 3:
Cas10d Myxacorys californica WJT36-NPBG1 (176-184)
N′-FGAIAAQLT-C′ (SEQ ID NO:78)
Cas10d Synechocystis sp. PCC 6803 (229-237)
N′-FGDVAVHLS-C′ (SEQ ID NO:79)

FIGS. 10A-10C provide diagram and graphical representations of the transposition efficiency by spacer and the effect of mismatches.

FIGS. 11A-11B provide diagram and graphical representations to demonstrate the effect of expressing additional Cas11d, Cas7d with extended spacers. Sequences on FIG. 11A, top line nucleotide sequence, amino acid sequence under that, second amino acid sequence, bottom nucleotide sequence:

From top to bottom:

(SEQ ID NO: 80)
CTTTTTTCCCAAGGAAATATTGTTATGACCGAAAAATTGAAACTGACTAA
A
(SEQ ID NO: 81)
LFSQGNIVMTEKLKLTK
(SEQ ID NO: 82)
VWAAGDSNMEQQLELTQ
(SEQ ID NO: 83)
GTATGGGCAGCAGGAGATTCAAACATGGAACAGCAATTGGAGCTAACTCA
G

FIG. 12 provides graphical representations to demonstrate the effect of having mismatches at the extended region of the spacers.

FIGS. 13A-13B provide diagram and graphical representations to demonstrate the TGT/ACA end sequence is not universally conserved in Tn7-like transposons.

FIG. 14 provides a diagram representation that demonstrates the convergent evolution of dual pathway lifestyle of CAST elements.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step, may be made without departing from the scope of the disclosure.

Unless defined otherwise, all technical and scientific terms used herein have the same meaning as commonly understood by one of ordinary skill in the art to which this invention belongs.

Every numerical range given throughout this specification includes its upper and lower values, as well as every narrower numerical range that falls within it, as if such narrower numerical ranges were all expressly written herein.

As used in the specification and the appended claims, the singular forms “a” “and” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about” it will be understood that the particular value forms another embodiment. The term “about” in relation to a numerical value is optional and means for example +/−10%.

This disclosure includes every amino acid sequence described herein and all nucleotide sequences encoding the amino acid sequences. Polynucleotide and amino acid sequences having from 50-99% similarity, inclusive, and including and all numbers and ranges of numbers there between, with the sequences provided here are included in the invention. All of the amino acid sequences described herein can include amino acid substitutions, such as conservative substitutions, that do not adversely affect the function of the protein that comprises the amino acid sequences, and may include other components, as further described below.

The disclosure includes all polynucleotide and all amino acid sequences that are identified herein by way of a database entry. Such sequences are incorporated herein as they exist in the database on the effective filing date of this application or patent.

The present disclosure provides recombinant, isolated, and/or modified configurations of Tn7-like elements. The disclosure includes modifications and use of a family of CAST elements formed by cooption of a type I-D CRISPR-Cas system, an unusual subtype with features of type I and type III effector systems. The disclosure reveals useful attributes of the I-D system that allow reduced system components and engineering embodiments stemming from flexibility with guide RNA design. The present disclosure also reveals cyanobacteria as a reservoir of diverse Tn7-like elements showing multiple examples of transposon targeting formed by convergent evolution, and provides modifications of such system for use in DNA editing.

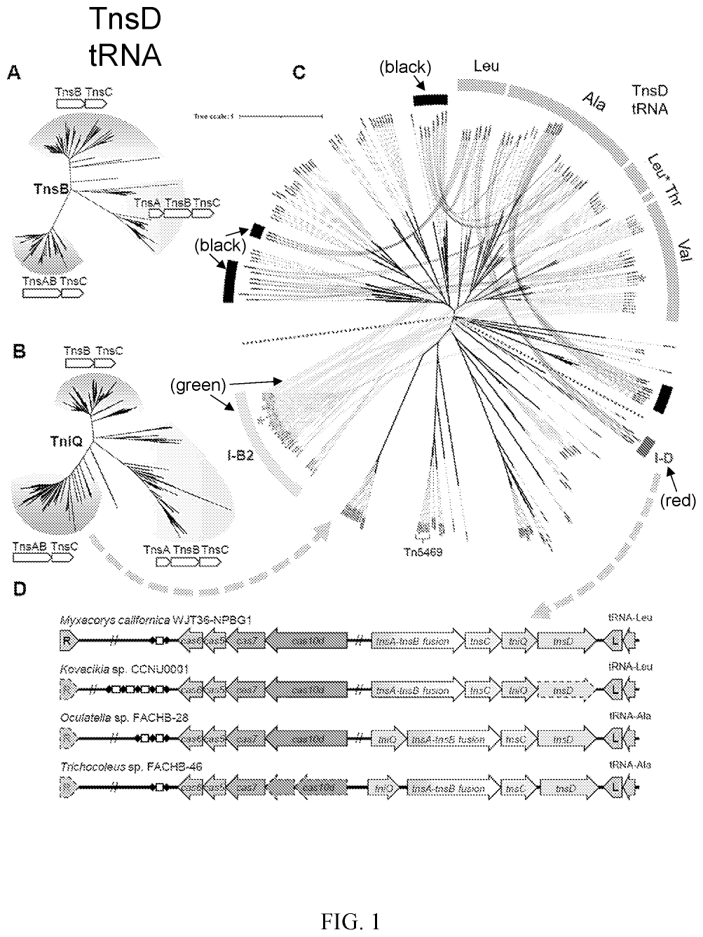

The disclosure relates to I-D CAST systems, representative examples of which are shown in FIG. 1, and further described herein. The described systems relate in part to the proteins described below, and modifications of the systems. In non-limiting embodiments, isolated or recombinantly expressed proteins of the disclosure comprise amino acid sequences or proteins that are expressed by Myxacorys californica WJT36-NPBG1 or any protein that has at least 50% sequence identity with a Myxacorys californica WJT36-NPBG1 protein. The disclosure includes the amino acid sequences in the following database entries, and all polynucleotides encoding them:
TnsAB: MBW4418955.1
TnsC: MBW4418954.1
TniQ: MBW4418953.1
TnsD: MBW4418952.1
Cas6: MBW4418981.1
Cas5: MBW4418980.1
Cas7: MBW4418979.1
Cas10: MBW4418978.1

In embodiments, the disclosure provides a system for use in DNA modification. A described system may be referred to herein as an McCAST system. The system comprises recombinantly produced or isolated CAST proteins, and may exclude Cas6, also referred to herein as Cas6d. The proteins are provided with a guide RNA that has a flexible design that allows modifications, including but not necessarily limited to the 3′ end of a guide RNA, such as a processed guide RNA that is functional with the described proteins. A functional guide RNA is a guide RNA that directs a system comprising the described proteins to a selected target site in DNA. The systems include, in addition to the guide RNA, a TnsC protein; a TnsD protein; a TniQ protein; a fusion protein comprising TnsA and TnsB proteins, a Cas5 protein, Cas7 protein, and a Cas10 protein. In embodiments, the Cas10 protein is inactivated.

In certain embodiments, including but not necessarily limited to a system that does not use a Cas6 protein, the system comprises a ribozyme component. The ribozyme component is capable of processing a precursor of the guide RNA. The ribozyme component may be provided as a component of a precursor of a processed guide RNA, or the ribozyme may be provided as a separate polynucleotide. An expression vector can also be used to provide the ribozyme. The type of ribozyme is not particularly limited, provided it cleaves at the 5′ and 3′ of a crRNA. The ribozyme component may exhibit self-cleaving activity if the ribozyme is a component of a polynucleotide that comprises a guide RNA sequence. In embodiments, the ribozyme is a hammerhead ribozyme, a hairpin ribozyme, or a hepatitis delta virus (HDV) ribozyme.

Regardless of the presence or absence of Cas6 in the described systems, the present disclosure demonstrates that modifications of the guide RNA can be made. Such modifications include but are not limited to extending its length, including but not limited to its 3′ end, relative to the length of a naturally occurring I-D CAST system. The modified guide RNA functions, or exhibits improved function, in a described system. In non-limiting embodiments, the guide RNA can include, for example, a functional RNA segment such as any of the described ribozyme segments, or binding sites for proteins or polynucleotides. In embodiments the guide RNA includes one or more binding sites for one or more proteins, which can include but are not necessarily limited to proteins with or without enzymatic activity. In embodiments the RNA includes one or more binding sites for one or more proteins that are any of DNA or RNA polymerases, helicases, telomerases, topoisomerases, histone modifiers, splicing factors, Pumilio proteins, viral proteins, transcription factors, or adapter proteins. In an embodiment, the guide RNA is modified such that it is a prime editing guide RNA (pegRNA). The pegRNA carries a primer binding site (PBS) that allows a reverse transcriptase to create a primer, which anneals to the DNA template near the target site. The reverse transcriptase extends the primer, using the target DNA strand as a template, to create a new DNA sequence that includes addition of specific nucleotides that match the desired edit. In this configuration, any suitable reverse transcriptase may be provided in trans in a system of this disclosure, or may be encoded by an expression vector. Thus, the disclosure includes modified guide RNAs that have a sequence that can bind to another polynucleotide, including but not necessarily limited to an RNA or DNA primer.

In embodiments, the guide RNA can be modified to include MS2 bacteriophage coat protein binding sites. In embodiments, the guide RNA forms two MS2 loops. The sequence that forms the loops in a non-limiting embodiment comprises the sequence acaugaggaucacccaugu (SEQ ID NO:84). Two copies of this sequence may be present and spaced apart such that the MS2 protein binds to the guide RNA. In an embodiment, the MS2 protein comprises or consists of the MS2 protein sequence available under UniProt database P03612 CAPSD_BPMS2. Using the MS2 binding sites within the guide RNA allows a protein that is modified to comprise a segment that comprises the MS2 protein to bind to the guide RNA. The disclosure therefore includes combining any protein that is modified to include an MS2 protein segment such that it associates with a guide RNA that contains MS2 protein binding sites. In one non-limiting embodiment, such as with a pegRNA format, the system can include a reverse transcriptase that may be modified to include MS2 RNA binding sequences, and thus the system may be used for prime editing.

Any protein described herein can be modified to include linking amino acids, or cellular trafficking signals, such as a nuclear localization signal. In embodiments, the modification comprises a nuclear localization sequence (NLS) that functions in trafficking the modified protein to the nucleus of a cell. Suitable NLS sequence are known in the art and can be adapted for use with the proteins described herein when given the benefit of the present disclosure. In embodiments, proteins described herein may be expressed from a coding sequence that includes a ribosomal skipping sequence. Ribosomal skipping sequences are known in the art and include, in non-limiting embodiments, the ribosomal skipping peptides T2A, P2A, E2A, and F2A.

In embodiments, use of a described system exhibits at least one improved property, relative to the same property of a control system. In embodiments, a control system uses an unmodified guide RNA, and/or includes a Cas6 protein. In embodiments, the disclosure facilitates an increase of transposition efficiency relative to a control, such as transposition from a chromosome to a plasmid, of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, fold greater than a control value. Similar transposition efficiency can be determined for transposition events where the transposition comprises transposing an element in cis, e.g., transposition from one location in a chromosome to a different location in the same chromosome. In a embodiments, detectable markers and selection elements can be used. In embodiments, transposition frequency can be measured, for example, by a change in expression in a reporter gene. Any suitable reporter gene can be used, non-limiting examples of which include adaptations of standard enzymatic reactions which produce visually detectable readouts. In embodiments, adaptations of β-galactosidase (LacZ) assays are used. In embodiments, transposition of an element from one chromosomal location to another, or from a plasmid to a chromosome, or from a chromosome to a plasmid, results in a change in expression of a reporter protein, such as LacZ. In embodiments, use of a system described herein causes a change in expression of LacZ, or any other suitable marker, in a population of cells. In embodiments, transposition efficiency is determined by measuring the number of cells within a population that experience a transposition event, as determined using any suitable approach, such as by reporter expression, and/or by any other suitable marker and/or selection criteria. In embodiments, the disclosure provides for increased transposition, such as within a population of cells, relative to a control. As described above, the control can be any suitable control, such as a reference value, or any value using a control experiment with proteins that have different modifications. In embodiments, the reference value comprises a standardized curve(s), a cutoff or threshold value, and the like. In embodiments, transposition efficiency comprises use of a system of this disclosure to transpose all or a segment of DNA from one location to another within the same or separate chromosomes, from a chromosome to a plasmid, or from a plasmid or other DNA cargo to a chromosome. In embodiments, transposition efficiency is greater than a control value obtained or derived from transposition efficiency using the described system.

The described systems may also include a DNA cargo sequence for use in insertion into a DNA substrate. The DNA cargo sequence can include left and right end transposon sequences. The transposon left and right end sequences may also be inserted with a DNA cargo. The DNA cargo sequence is inserted into a DNA substrate by cooperation of the described proteins and the guide RNA to produce the DNA editing. Those skilled in the art will be able to understand the terms “left” and “right” transposon sequences, and recognize such sequences. In embodiments, the system is targeted via a described guide RNA to a sequence in a chromosome in a eukaryotic cell, or to a DNA extrachromosomal element in a eukaryotic cell, such as a DNA viral genome. Thus, the disclosure includes modifying eukaryotic chromosomes, and eukaryotic extrachromosomal elements, such as DNA in any organelle. Accordingly, the type of extrachromosomal elements that can be modified according to the presently described compositions and methods are not particularly limited. Accordingly, instead of transposing an existing segment of a genome in the manner in which transposons ordinarily function, the disclosure provides for insertion of DNA cargo that can be selected by the user of the system. The DNA cargo may be provided, for example, as a circular or linear DNA molecule. The DNA cargo can be introduced into the cell prior to, concurrently, or after introducing a system of the disclosure into a cell. The sequence of the DNA cargo is not particularly limited, other than a requirement for suitable right and left ends that are recognized by proteins of the system. The right and left end sequences that are required for recognition are typically from about 90-150-bp in length. The minimum length of the DNA cargo can be 700 bp to 120 kb. The disclosure provides for insertion of a DNA cargo without making a double-stranded break, and without disrupting the existing sequence, except for residual nucleotides at the insertion site, as is known in the art for transposons. In embodiments, the insertion of the DNA cargo occurs at a position that is 31, 32, 33, 34, 35, 36, 37, 38, 39, 40, 41, 42, or 43 nucleotides from a protospacer in the target (e.g., chromosome or plasmid) sequence.

In embodiments, the compositions and methods of this disclosure are functional in a heterologous system. “Heterologous” as used herein means a system, e.g., a cell type, in which one or more of the components of the system are not produced without modification of the cells/system. A non-limiting embodiment of a heterologous system is any bacteria that is not Myxacorys californica WJT36-NPBG1. In embodiments, a representative and non-limiting heterologous system is any type of E. coli. A heterologous system also includes any eukaryotic cell.

In embodiments, a system of this disclosure is introduced into cells using, for example, one or more expression vectors, or by direct introduction of ribonucleoproteins (RNPs). In embodiments, expression vectors comprise viral vectors. In embodiments, a viral expression vector is used. Viral expression vectors may be used as naked polynucleotides, or may comprises any of viral particles, including but not limited to defective interfering particles or other replication defective viral constructs, and virus-like particles. In embodiments, the expression vector comprises a modified viral polynucleotide, such as from an adenovirus, a herpesvirus, or a retrovirus, such as a lentiviral vector. In embodiments, a baculovirus vector may be used. In embodiments, any type of a recombinant adeno-associated virus (rAAV) vector may be used. In embodiments, a recombinant adeno-associated virus (rAAV) vector may be used. rAAV vectors are commercially available, such as from TAKARA BIO® and other commercial vendors, and may be adapted for use with the described systems, given the benefit of the present disclosure. In embodiments, for producing rAAV vectors, plasmid vectors may encode all or some of the well-known rep, cap and adeno-helper components. In certain embodiments, the expression vector is a self-complementary adeno-associated virus (scAAV). Suitable ssAAV vectors are commercially available, such as from CELL BIOLABS, INC.® and can be adapted for use in the presently provided embodiments when given the benefit of this disclosure. In embodiments, one or more expression vectors of the disclosure comprise at least one of TnsC, TnsD, TniQ, TnsATnsB, Cas5, Cas7, and Cas10 genes.

Further modification of this approach can include expression and isolation of the proteins required for this process and carrying out some or all of the process in vitro to allow the assembly of novel DNA substrates. These DNA substrates can subsequently be delivered into living host cells or used directly for other procedures. Thus, the disclosure includes compositions, methods, vectors, and kits for use in the present approach to DNA editing.

In embodiments, a system of this disclosure is administered to an individual in a therapeutically effective amount. In embodiments, a therapeutically effective amount of a composition of this disclosure is used. The term “therapeutically effective amount” as used herein refers to an amount of an agent sufficient to achieve, in a single or multiple doses, the intended purpose of treatment. The amount desired or required will vary depending on the particular compound or composition used, its mode of administration, patient specifics and the like. Appropriate effective amounts can be determined by one of ordinary skill in the art informed by the instant disclosure using routine experimentation. For example, a therapeutically effective amount, e.g., a dose, can be estimated initially either in cell culture assays or in animal models. An animal model can also be used to determine a suitable concentration range, and route of administration. Such information can then be used to determine useful doses and routes for administration in humans, or to non-human animals. A precise dosage can be selected by in view of the patient to be treated. Dosage and administration can be adjusted to provide sufficient levels of components to achieve a desired effect, such as a modification in a threshold number of cells. Additional factors which may be taken into account include the particular gene or other genetic element involved, the type of condition, the age, weight and gender of the patient, desired duration of treatment, method of administration, time and frequency of administration, drug combination(s), reaction sensitivities, and tolerance/response to therapy. In certain embodiments, a therapeutically effective amount is an amount that reduces one or more signs or symptoms of a disease, and/or reduces the severity of the disease. A therapeutically effective amount may also inhibit or prevent the onset of a disease, or a disease relapse. In embodiments, cells modified according to this disclosure are administered to an individual in need thereof in a therapeutically effective amount. In embodiments, the disclosure includes obtaining cells from an individual, modifying the cells ex vivo using a system as described herein, and reintroducing the cells or their progeny into the individual or an immunologically matched individual for prophylaxis and/or therapy of a condition, disease or disorder, or to treat an injury, trauma or anatomical defect. In embodiments, the cells modified ex vivo as described herein are autologous cells. In embodiments, the cells are provided as cell lines. In embodiments, the cells are engineered to produce a protein or other compound, and the cells themselves and/or the protein or compound they produce is used for prophylactic or therapeutic applications.

In embodiments, the disclosure comprises providing a treatment to an individual in need thereof by introducing a therapeutically effective amount a composition of this disclosure, or modified cells as described herein to the individual, wherein the cells comprising the DNA insertion treats, alleviates, inhibits, or prevents the formation of one or more conditions, diseases, or disorders. In embodiments, the cells are first obtained from the individual, modified according to this disclosure, and transplanted back into the individual. In embodiments, allogenic cells can be used. In embodiments, the modified eukaryotic cells can be provided in a pharmaceutical formulation, and such formulations are included in the disclosure.

With respect to the foregoing description, it will be recognized by those skilled in the art that Tn7-like elements are abundant in cyanobacterial genomes, including most subtypes that are capable of RNA-guided transposition. The discussion above and the examples below describe a novel cooption of a type I-D CRISPR-Cas system for RNA-guided transposition and insertion of DNA cargoes. The presently described mechanism used for coopting the CRISPR-Cas system is distinct from the other well-studied examples. The major interface between the TniQ protein and I-F3 Cascade is via Cas6f while in the I-D McCAST system described herein, and as discussed above, the Cas6d protein is not essential for guide RNA-directed transposition. Both the type I-F3 and I-D systems show a low level of off-site targeting and tight orientation control. Unlike the I-F3 CAST elements, the presently described I-D McCAST element maintains a PAM preference found in the canonical CRISPR-Cas system where it was likely derived, and an anti-PAM property with the I-D system is described. Maintaining the PAM system is advantageous for limiting targeting into the CRISPR array, an issue found with the type I-F3 systems that show extensive PAM ambiguity. Flexibility in accommodating a variety of guide RNA lengths and independence from Cas6 through guides that are auto-processed with ribozymes facilitates the described modifications of the I-D system to new heterologous hosts. The ability to extend the guide RNA also allows described modifications, which may be appended to the PAM distal region of the guide.

Without intending to be constrained by any particular interpretation, it is considered that the presently described type I-D CRISPR-Cas system includes certain features that suggest a more recent CRISPR-Cas cooption event than other systems. The type I-F3 CAST systems are more diverged from the canonical I-F1 systems than is found with the type I-D CAST and canonical systems. Maintenance of a robust PAM system with type I-D CAST is also consistent with the interpretation that cooption was more recent. The present disclosure describes a I-D system that is 56% identical to McCAST with its central Cas10d protein (MBD1847458.1) was found. A type I-D CAST element that appears to maintain the Cas1,2,4 adaptation system (Cyanothece sp. PCC 7425, accession number NC_011884) is also described.

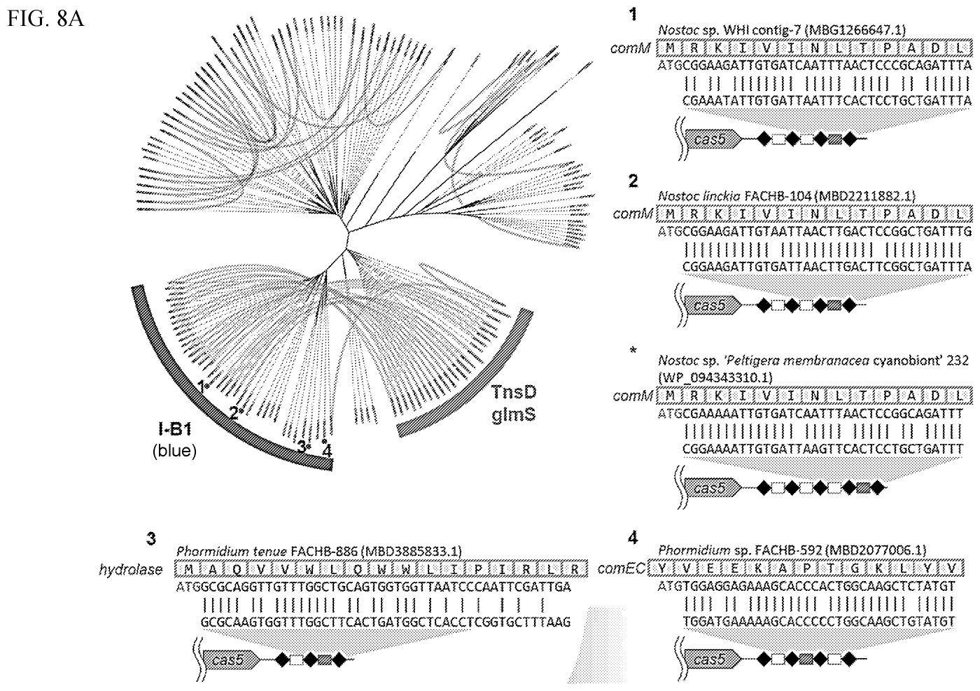

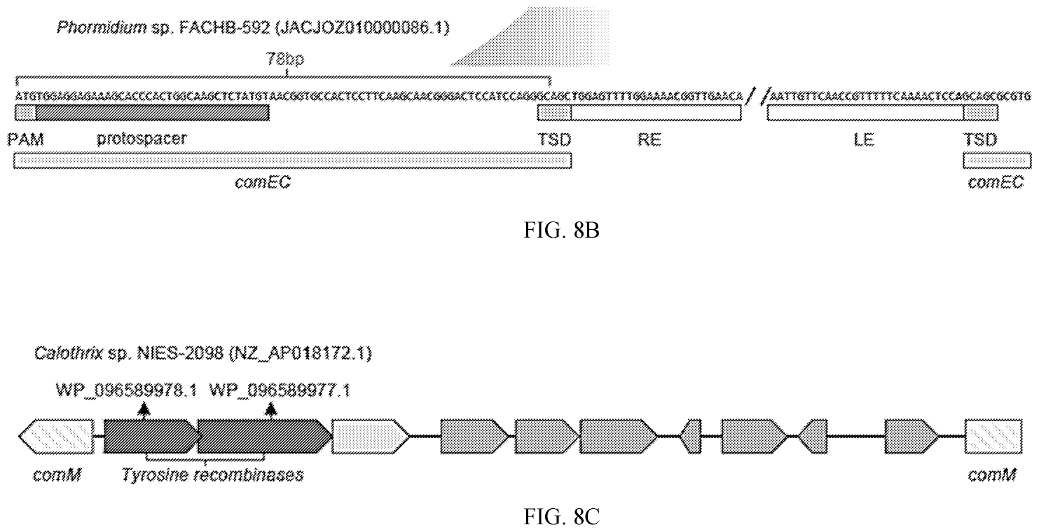

Multiple groups of Tn7-like elements converged on the strategy of using separate TniQ family proteins, including the type I-B1 and I-B2 systems and the type I-D system described herein (FIG. 14). In another example of Tn7-like elements using separate TniQ family proteins to allow two transposition pathways, the I-F3 TniQ-Cascade system was coopted by a family of Tn7-like elements with a TniQ targeting an attachment site downstream of parE. The type I-F3 and V-K CAST systems converged on the strategy where separate classes of guide RNA evolved to allow a targeting system that recognizes a conserved attachment site in the chromosome and a separate series of guides that targets mobile elements capable of cell-to-cell transfer. This analysis indicates that the I-B1 family has undergone a similar transition in re-evolving dual pathways using different guide RNAs with a single TniQ protein (FIGS. 8A-C, FIG. 14).

FIG. 14 displays convergent evolution of dual pathway lifestyle of CAST elements. Similar to Tn7 (top), all currently known CAST convergently evolved dual pathway lifestyles. The gene arrangement of Tn7 and some representatives of the five proven types of CAST elements are shown on the left; the target selectors for the two transposition pathways are shown on the right. Some use two different TniQ target selectors; some use only TniQ-Cas but with two different kinds of spacers/crRNAs. The type I-D CAST in this disclosure is outlined with a rectangle. TniQ proteins are indicated with a small circle and the TniQ-domain-containing protein TnsD with an oval; Cas proteins and chromosome-targeting spacers/crRNAs are shown.

The following examples are presented to illustrate the present disclosure. They are not intended to be limiting in any matter. The examples utilized the following materials and methods.

Escherichia coli strains (Table 1) were grown in lysogeny broth (LB) or on LB agar supplemented with the following concentrations of antibiotics when appropriate: 100 g/mL carbenicillin, 10 g/mL gentamicin, 30 g/mL chloramphenicol, 8 g/mL tetracycline, 50 g/mL kanamycin, 50 g/mL spectinomycin, 20 g/mL nalidixic acid, 100 g/mL rifampicin, 50 g/mL X-gal.

TABLE 1
Strains used in this disclosure.

| Strain | Genotype |
|---|---|
| BW27783 | F⁻, Δ(araD-araB) 567, ΔlacZ4787(::rrnB-3), λ-, Δ(araH-araF)570(::FRT), ΔaraEp- |
| | 532::FRT, φPcp8-araE535, rph-1, Δ(rhaD-rhaB)568, hsdR514 |
| BW20767 | F⁻, RP4-2(Km::Tn7, Tc::Mu-1), ΔuidA3::pir⁺, leu-163::IS10, recA1, creC510, |
| | hsdR17, endA1, thi |
| CW51 | F⁻, ara⁻, arg-, Δ(lac-pro)XIII, nal^{R}, rif^{R}, recA56 |
| PO677 | BW27783 attTn7:mTn7-miniMcCAST(KanR) |
| PO788 | PO677 pOPO717 (McCAST Cascade operon and lacZ spacer 5 under arapBAD |
| | control), pOPO636 (TnsABCQ under lac control) |
| PO619 | BW27783 lacZ⁺ |
| PO704 | BW20767 pOPO701(donor plasmid with mini-transposon of McCAST and an R6K |
| | origin of replication) |

Strain P0677 was constructed with a mini McCAST element in the chromosome at the neutral attTn7 position within a mini Tn7 element as described previously. A Lac⁺ derivative of BW27783, P0619, was constructed by using P1 transduction to move the wild type lac allele from wild type E. coli K-12 (CGSC #: 4401). Strain P0704 was used for delivery of a conditional replicon and oriT (RP4) containing pOPO701 vector with the mini McCAST element from the Pir⁺ donor strain BW20767 which encodes the RP4 conjugation machinery. Standard molecular cloning techniques were used to make the vectors described in supplementary Table 2 according to the vendor instructions. The biomass of Myxacorys californica WJT36-NPBG1 was donated by Dr. Nicole Pietrasiak. The genomic DNA was extracted with DNeasy PowerLyzer Microbial Kit (QIAGEN) as described before.

Annotated protein fasta files, genomic sequences, and feature tables of cyanobacteria were downloaded from National Center for Biotechnology Information (NCBI) FTP site. In total, there were 2,163 genomes for analysis. Profile HMMs associated with TnsA (PF08722, PF08721), TnsB (PF00665), TnsC (PF11426, PF05621), TniQ (PF06527) downloaded from the European Bioinformatics Institute (EMBL-EBI) Pfam database, were used for detecting homologs with hmmsearch (HMMER3). Candidate proteins were grouped into tnsBC operons, and each operon was then grouped with its neighboring tnsA and tnsQ into one transposon functional unit. The tnsA and tniQ adjacent to more than one tnsBC operon are allocated to the closest one. Only those with at least one tnsA or tnsQ are collected. The TnsB and TniQ proteins were aligned with MUSCLE.

Similarity trees were made with FastTree using WAG evolutionary model and the discrete gamma model with 20 rate categories as previously described. The visualization of the trees and coloring was done with iTOL (Interactive Tree Of Life).

The frequency of transposition was monitored in a large pool of independent transformants, as described previously. Briefly, vectors encoding the core transposase genes (TnsABCQ/TnsABC with lactose induction) and target selection genes (Cascade operon, crRNA/TnsD with arabinose induction) were co-transformed into cells (BW27783 background) carrying an F plasmid derivative with the target sequence and the mini-McCAST element (Kanamycin resistance gene flanked by left and right McCAST transposon ends) on a donor plasmid. Plates were grown overnight, and hundreds of transformants were washed off the plate in LB media, pelleted, washed twice with M9 minimal media, and finally resuspended to O.D. 0.6 in M9 minimal media supplemented with 0.2% w/v maltose, required antibiotics, 0.2% w/v arabinose, and 0.1 mM IPTG for induction. After 18 hours of incubation with shaking at 30° C., 0.5 ml of the donor cells was spun down, washed twice with LB, and resuspended into 0.5 ml LB supplemented with 0.2% w/v glucose for recovery with shaking at 37° C. for 30 min. To monitor transposition from the donor plasmid into the F plasmid target, donor cells were then mixed with mid-log recipient cells (CW51) in LB supplemented with 0.2% w/v glucose at a ratio of 1:5 donor:recipient and incubated with gentle agitation for 90 minutes at 37° C. to allow mating. After incubation, cultures were vortexed, placed on ice, then serially diluted in LB 0.2% w/v glucose and plated on LB supplemented with required antibiotics for selecting CW51 recipient cells for transconjugants g/mL nalidixic acid, 100 g/mL rifampicin, 50 g/mL spectinomycin, 50 g/mL X-gal, with or without 50 g/mL kanamycin to sample the entire transconjugant population or select for transposition respectively. Plates were incubated at 37° C. for 24 hours before colonies were counted. For testing the effects of expressing additional Cas11d and Cas7d, pOPO808 or empty vector control pBBR-GenR-ara was co-transformed with the other transposition gene expression vectors, with 10 g/mL gentamycin supplemented into LB agar and induction M9 minimal media in the following step.

To confirm the target site duplication expected with transposition, transposon junctions from insertions in the lacZ gene (guided by lacZ spacer 1) were amplified by colony PCR with primer pairs JEP2257+ JEP2901 and JEP1597+ JEP2903 (Table 2) and subjected to Sanger DNA sequencing. Illumina sequencing was used to map the total insertions from F plasmids from transconjugants. Transconjugants were collected, and F plasmid DNA was isolated using the ZR BAC DNA Miniprep Kit. Insertions were mapped with BBtools (BBMap—Bushnell B.—sourceforge.net/projects/bbmap/).

TABLE 2
Oligonucleotide primers used in this disclosure

| name | Primer and description | Sequence |
|---|---|---|
| JEP2257 | Amplify left end junction | 5′-CCGCGCTGTACTGGAGGCTGAAGTT-3′ (SEQ ID NO: 85) |
| JEP2901 | Amplify left end junction | 5′-TTGGTCTCTTCAGCTCCTCATGTAAAAGTGTCTTCAAA- |
| | | 3′ (SEQ ID NO: 86) |
| JEP1597 | Amplify right end junction | 5′-CAGCGACCAGATGATCAC-3′ (SEQ ID NO: 87) |
| JEP2903 | Amplify right end junction | 5′-TTGGTCTCTCCAATTACCAGCACCATGATCTTTATAA-3′ |
| | | (SEQ ID NO: 88) |
| JEP3375 | Making PAM library | 5′-GTTGCTCTTCAAGAGTTGCCCGGCGCTCTCCGGCTGCC |
| | | CGGCTTCCATTCAGGTCGAG-3′ (SEQ ID NO: 89) |
| JEP3376 | Making PAM library | 5′-GTTGCTCTTCATCTGGCTCACAGTACGCGTAGTGCNN |
| | | NNTGCAGAATCCCTGCTTCGT-3′ (SEQ ID NO: 90) |

A PAM library was constructed by PCR amplification of plasmid pBBR-GenR with JEP3375+ JEP3376, subsequent digestion with SapI and self-ligation. The plasmid PAM library was transformed into DH5α, pooled, and plasmid isolated for PAM screening. To screen PAM preference, the PAM library was electroporated into the P0788 (BW27783 with vectors carrying the transposition genes) and plated on LB agar supplemented with the appropriate antibiotics, and 0.1 mM IPTG, and 0.2% w/v arabinose for induction. After 17 hours of incubation at 37° C., the colonies were scrapped from the plates, and the plasmids extracted then retransformed into DH5α with electroporation for selecting those with insertions on LB agar supplemented with 50 g/mL kanamycin and 10 g/mL gentamycin. Each step of the process was repeated to ensure a library coverage greater than 80×. The plasmids with transposon insertions and the original PAM library were sent to Illumina sequencing for comparing their PAM compositions.

To monitor whether the TnsAB fusion protein of McCAST moves by cut-and-paste transposition or forms cointegrates, the following examples monitored vector backbone integration genetically following a mate-in transposition assay with an appropriate control. A donor plasmid carrying a mini-McCAST element and TetR marker on its backbone (pOP0701) was delivered by conjugation into recipient cells where the donor plasmid cannot replicate. Transposition by simple insertion or cointegrate formation could be assessed by monitoring whether the backbone TetR marker was retained after transposition in recipient cells. The recipient strain P0619 (Escherichia coli BW27783 lacZ⁺) was freshly transformed with vectors carrying transposition genes. Overnight cultures of the transformed recipient strain were diluted 50 times into induction media (LB, 0.1 mM IPTG, 0.2% (w/v) arabinose, required antibiotics), and grown to mid-log phase. In parallel, an overnight culture of the donor strain P0704 (BW20767 carrying pOPO701) was diluted 25 times into LB with appropriate antibiotics and grown to mid-log phase. The cultures of donor and recipient strains were spun down, washed with LB twice, and resuspended to O.D.600=10. The donor was then mixed with recipients in a ratio 1:5, 20 μl of each mixture was spotted on LB plate supplemented with 0.1 mM IPTG and 0.2% (w/v) arabinose. Conjugal mating was conducted at 30° C. for 2 hours. After mating, each spot was washed up with 3 ml LB medium, serial diluted, and plated on LB plates supplemented with appropriate antibiotics and X-gal. One hundred fifty white colonies (presumably on-lacZ transposition) were purified onto a fresh plate, then streaked on LB agar supplemented with tetracycline to test for cointegration of donor plasmid backbone. As a control, the experiment was repeated with different combinations of vectors carrying transposition genes (TnsABC+TniQ with or without a TnsA active site mutation, Cascade operon with and without target spacer) transformed into the recipient strain as described in the text.

Statistical details are listed in Figure Legends. When stated, experiments were performed with three biological replicates (n=3).

### Example 1

This example provides a description of diverse configurations of Tn7-like elements found in cyanobacteria.

This example surveyed 2,163 annotated cyanobacterial genomes on NCBI for Tn7-like transposons, defined as transposons with TnsB and TnsC and encoding either TnsA or TniQ family proteins, and found more than 800 Tn7-like transposons. Similarity trees of TnsB and TniQ subdivided candidate Tn7-like elements based on basic transposase architecture, elements without TnsA (i.e., only the TnsB transposase in addition to TnsC and TniQ), elements with separate TnsA and TnsB transposase proteins, or derivatives with TnsA and TnsB fused as the transposase (FIG. 1, panels A-B). Different types of CAST elements are found across all three branches of transposons and distinguished by transposase architecture. The clade that lacked a tnsA gene is predominated by type V-K CAST systems, elements with a separate tnsA gene include type I-B1 CAST elements, and the clade with the tnsAB fused transposase includes type I-B2 CAST elements.

To analyze TniQ diversity and CAST pathway acquisition, this example primarily focused on the clade with the fused TnsAB transposase (FIG. 1, panel C). Most transposons in the TnsAB clade found in tRNA attachment sites that based on similarity trees are likely recognized by a TnsD-like protein with an N-terminal TniQ domain (PF06527) and a C-terminal DNA binding domain (FIG. 1, panel C). These elements often also encoded a second TniQ protein. Based on the known behavior of Tn7-like elements to typically have a second pathway that targets mobile plasmids, this example examined the TniQ branch positions in elements encoding two TniQ proteins.

It was hypothesized that if the TnsD-like protein is for targeting transposition into the tRNA gene attachment site, the second TniQ encoded in the element is likely adapted for a targeting pathway facilitating the horizontal transfer of the element. This analysis revealed six prominent TniQ branches as putatively adapted as an alternative targeting pathway based on forming independently branching phylogenetic groups (marked with black, green, and red bars in FIG. 1, panel C). Two of the TniQ branches identified using this analysis consisted of proteins lacking C-terminal DNA binding domains, a feature common among known CAST systems (marked with green and red bars in FIG. 1, panel C). One such branch includes the recently validated type I-B2 CRISPR-coopting TniQ (green bar in FIG. 1, panel C); however, a second branch within this group of tRNA targeting elements was a group with a distinct branch of small TniQ family proteins (red bar in FIG. 1, panel C). Instead of possessing a type I-B2 CRISPR-Cas system, this small group associated with type I-D CRISPR-Cas systems suggests a new example of CRISPR-Cas cooption. The type I-D CRISPR-Cas associated transposons are closely related to type I-B2 PmcCAST in the core Tns proteins (˜48% a.a. sequence identity of concatenated TnsABCD). Multiple features of the associated type I-D CRISPR-Cas suggested that the system had been coopted for RNA-guided transposition.

Canonical type I-D CRISPR-Cas systems shares features common to both type I and type III CRISPR systems. Like other type I CRISPR-Cas systems, I-D systems have the signature Cas3 protein, but the Cas3 functional domains are separated in these systems as the Cas3′ protein and a Cas3″ functional domain is part of the Cas10 protein. Cas3′ contains the helicase domain for unwinding dsDNA allowing processive cleavage over long distances. The Cas3″ HD nuclease domain is part of the large subunit Cas10 protein, a protein typically associated with type III CRISPR-Cas systems. In addition, the Cas7 of type I-D CRISPR has a separate nuclease activity, enabling its Cascade complex to cut the target ssDNA strand at 6 nt intervals, much like how type III CRISPR-Cas Cascade cut target RNA.

Examining the architecture of the transposon-associated type I-D systems indicated they lack the cas3′ gene required for processive DNA cleavage found in canonical type I-D systems (FIG. 1, panel D), reminiscent of the loss of Cas3 in type I-F3 systems (FIGS. 9A-B). In addition, the transposon associated type I-D CRISPR systems maintain short CRISPR arrays and lack the spacer acquisition genes cas1, 2, 4 found in the canonical I-D system, which are convergent features shared by all known CAST families (FIG. 9A). Analysis of the Cas10d HD nuclease domain in the transposons reveals a change from the conserved HD residue that is normally required to coordinate a metal essential for nuclease activity (FIGS. 9A-C), whose importance was confirmed experimentally and structurally. This loss of active-site residues is reminiscent of nuclease-inactivating mutations in the Cas12k proteins in the type V-K CAST systems.

As shown in FIG. 1, bioinformatic analysis reveals a novel family of CAST. FIG. 1, panel A, displays a TnsB similarity tree of Tn7-like transposons in cyanobacteria. FIG. 1, panel B, displays a TniQ similarity tree of Tn7-like transposons in cyanobacteria. FIG. 1, panel C, displays a TniQ similarity tree of Tn7-like transposons with TnsAB fusion in cyanobacteria. The dashed line separates the tree into two parts, the top is mostly large TniQ (>450 a.a.), and the lower half is mostly small TniQ (<350 a.a.). TniQ proteins encoded in the same transposon are connected with curved lines. The tRNA-targeting TnsD are indicated with the specific tRNA indicated, tRNA-Leu* contains a group I intron. The type I-B1 CAST TniQ are indicated in green and type I-D CAST TniQ are indicated in red. The TniQ proteins of PmcCAST are marked with an asterisk. Another four prominent tRNA-targeting TnsD-associated secondary TniQ groups are marked with black bars. FIG. 1, panel D shows the gene configuration of four putative type I-D CAST, cargo genes are not shown for simplicity. Dashed outline means the transposon end cannot be found or the gene is a pseudogene. L: transposon left end; R: transposon right end.

As shown, FIGS. 9A-C display that transposon-associated type I-D CRISPR systems show features common to CAST systems. Specifically, FIG. 9A shows that the putative transcriptional regulator WYL, the helicase required for long-distance DNA cleavage Cas3′, the adaptation proteins Cas1, 2, 4 are missing in the transposon associated type I-D CRISPR system. FIG. 9B displays multiple alignment of McCAST Cas10d (MBW4418978.1) with the closest 50 Cas10d homologs from canonical type I-D systems and a previously characterized Cas10d from M. aeruginosa PCC9808 (WP_002791883.1), mutating the conserved HD residues of which abolishes the nuclease activity. Arrowhead indicates the conserved HD residues, and natural variant residues are labeled, all associated with putative type I-D CAST. FIG. 9C displays the alignment of transposon-associated Cas10d from M. californica WJT36-NPBG1 and canonical Cas10d from Synechocystis sp. PCC 6803 near HD nuclease active site residues. Two proteins are 46% identical. The active site residues (labeled red) of Synechocystis sp. PCC 6803 Cas10d were based on a previous structural study.

### Example 2

This example provides a description of McCAST as a type I-D CRISPR-guided transposon.

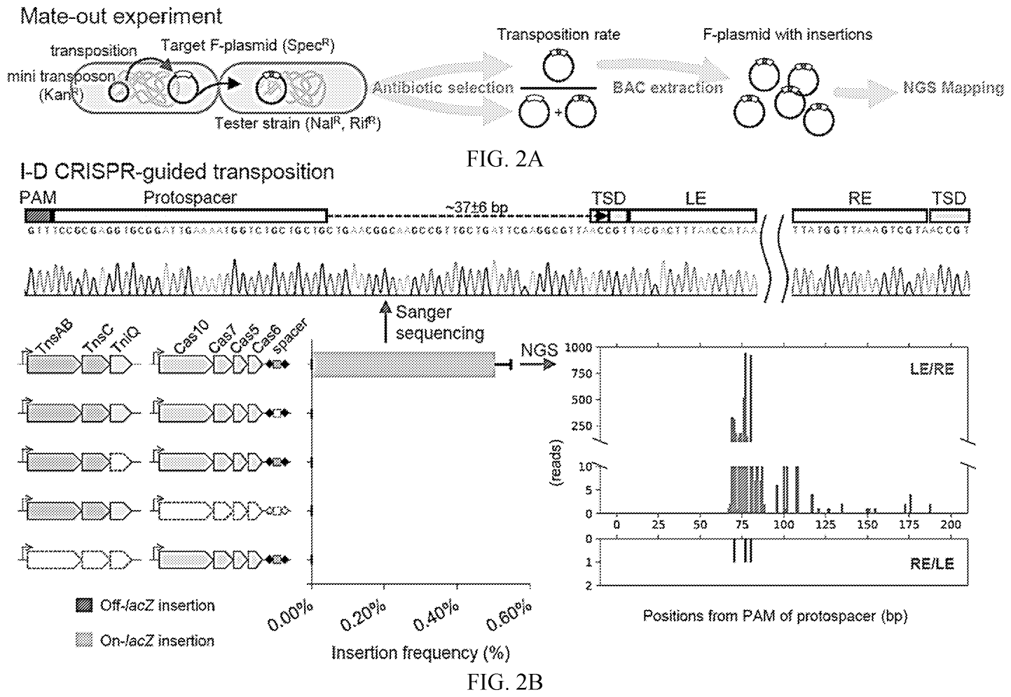

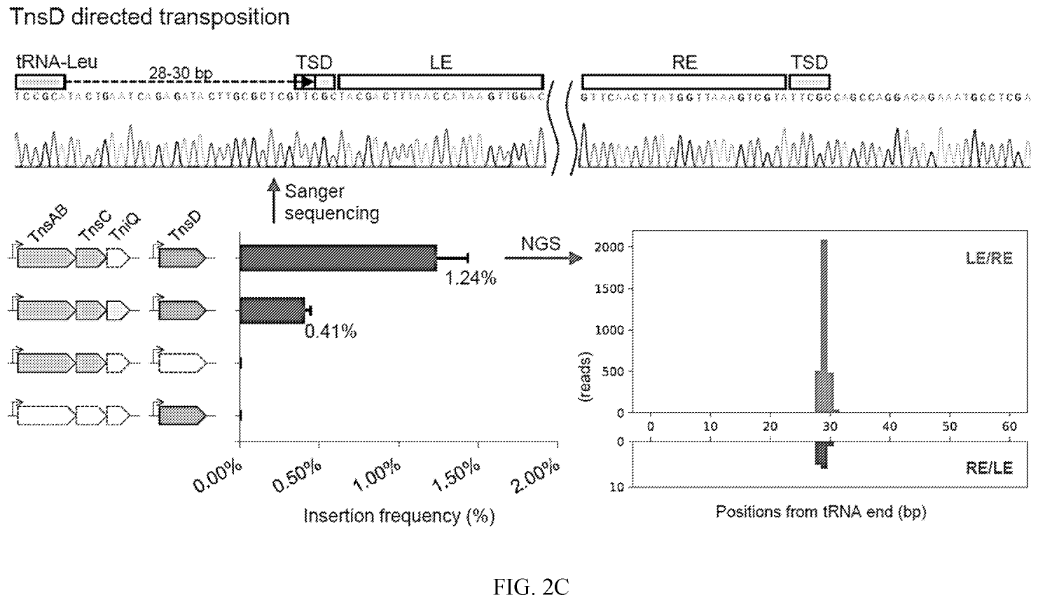

In this example, the type I-D CAST from Myxacorys californica WJT36-NPBG1 (McCAST) for experimental validation in a heterologous E. coli host were selected. McCAST is the only type I-D CAST where both ends of the element could be identified along with the characteristic target site duplication indicating transposition was used for the integration of the element. Additionally, all the CRISPR-associated and tranAposition genes were present and are not pseudogenes in this element (FIG. 1, panel D). RNA-guided transposition was tested in the heterologous E. coli host using a mate-out assay. In this example, a mini-McCAST transposon with the cis-acting transposon ends flanking an antibiotic resistance determinant was situated on a donor plasmid and a lacZ gene maintained on an F plasmid derivative as a transposition target (FIG. 2A). The cas and transposase genes were expressed from separate plasmids. The native single spacer array downstream of the cas operon was replaced with restriction sites for cloning and expressing candidate spacers. A spacer targeting the F plasmid-encoded lacZ gene was used for the transposition assay, and this example used a GTT protospacer adjacent motif (PAM) known to be favored in many type I-D CRISPR systems.

After inducing expression of the system, RNA-guided transposition events were detected and quantified by using conjugation to transfer F plasmids into a tester strain. Transposition assays indicated that the McCAST type I-D CRISPR-Cas was capable of guide RNA programmable transposition (FIG. 2B). RNA-guided transposition only occurs when the lacZ targeting spacer and the Cascade and TnsABCQ proteins are expressed. In this example, on-target and off-target transposition events were roughly estimated with LacZ activity (i.e. blue/white screen with X-Gal). Greater than 99% of the insertions render the F-plasmid LacZ-indicating a high level of guide RNA targeting. RNA-guided transposition was further verified by Sanger sequencing, showing the 5 bp target site duplication at the transposon ends (FIG. 2B). NGS mapping of F plasmids targets showed that the insertions are concentrated 75±6 bp downstream from the GTT PAM. Deep sequencing also allowed for visualization of a small fraction of insertions trailing downstream from the preferred site, something not observed with other CAST subtypes. Consistent with other Tn7-related transposons, insertion events also show the expected orientation bias, with >99% of insertions having transposon left end adjacent to the target sites.

The second, larger TniQ (TnsD) was predicted to target transposition into the tRNA-Leu attachment site in M. californica WJT36-NPBG1 based on the informatics analysis presented above. To confirm this prediction, this example constructed a target F-plasmid carrying a tRNA-Leu gene from M. californica WJT36-NPBG1 and a vector carrying the tnsD gene. It was found that the TnsD protein can direct insertions downstream of the tRNA-Leu gene at the position found natively in the M. californica genome (FIG. 2C). This pathway requires only TnsABC and TnsD. It was found that the expression of TniQ reduces the efficiency of the tRNA-targeting pathway, indicating the two TniQ family proteins may interfere with each other. Compared to the RNA-guided transposition events, the TnsD-guided insertions are more precise; almost all insertions are at 29±1 bp after the target tRNA-Leu.

In the type I-D McCAST system activity can vary between protospacers. Transposition rates varied when eight spacers in lacZ were randomly selected and tested, all with the predicted GTT PAM (FIG. 10A). The distribution of insertions fell within the range of 75±6 bp downstream of the start of GTT PAM and almost all in a single orientation (FIG. 10B). The experiment confirmed the programmability of McCAST.

To explore any differences from other CAST systems and canonical I-D CRISPR-Cas systems, mismatch tolerance was examined. Previous structural work with type I systems indicates that every 6^{th} position in the R-loop is flipped out and does not contribute to the specificity of the protospacer. It was found that a spacer with mismatches at every sixth position showed no reduction in transposition efficiency (FIG. 0C). Mismatches were not tolerated in the seed region and seed proximal region of the spacer. Consecutive 5 bp mismatches at any of the seed-proximal five Cas7 binding sites impairs transposition as much as the scrambled spacer control. Only mismatches at the most distal region where the most distal Cas7 is expected (31-35 bp) showed substantial transposition compared to controls (FIG. 10C).

FIGS. 2A-C display an in vivo transposition assay of the type I-D CRISPR-guided pathway and TnsD mediated tRNA-targeting with McCAST in E. coli. FIG. 2A displays a cartoon representation of the mate-out assay strategy (plasmids expressing transposon and Cas function omitted for clarity). FIG. 2B (left) displays the frequency of McCAST transposition into F-lacZ with different genetic backgrounds. In the diagram, trials of the experiment missing genes or the spacer are shown with dashed outlines. Data are shown as mean+SD, n=3. No off-lacZ insertions were detected, <0.1%. FIG. 2B (top) displays Sanger sequencing of an on-target insertion. (TSD: target site duplication, LE: left end, RE: right end). FIG. 2B (right) displays type I-D CRISPR-guided insertion distribution revealed through deep sequencing. Bars are as indicated for insertions with their left ends proximal to the lacZ protospacer; and insertions with right ends proximal to the lacZ protospacer (note differences in scale). FIG. 2C (left) displays the frequency of McCAST transposition into F-tRNA-Leu gene with different genetic backgrounds. FIG. 2C (top) displays Sanger sequencing of an on-target insertion. FIG. 2C (right) displays the insertion distribution of TnsD-guided transposition revealed through deep sequencing.

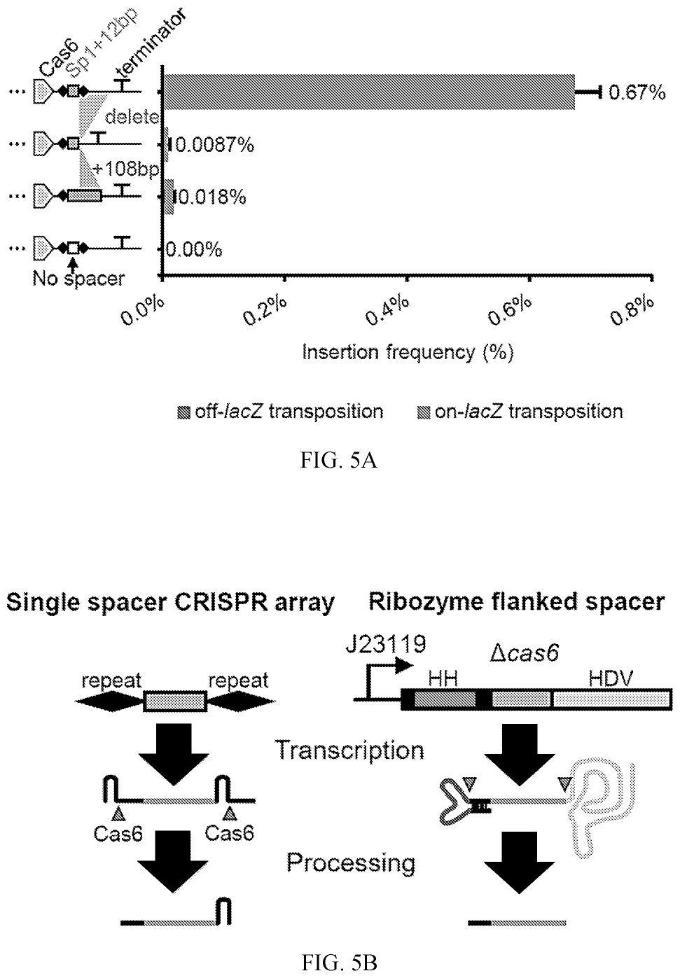

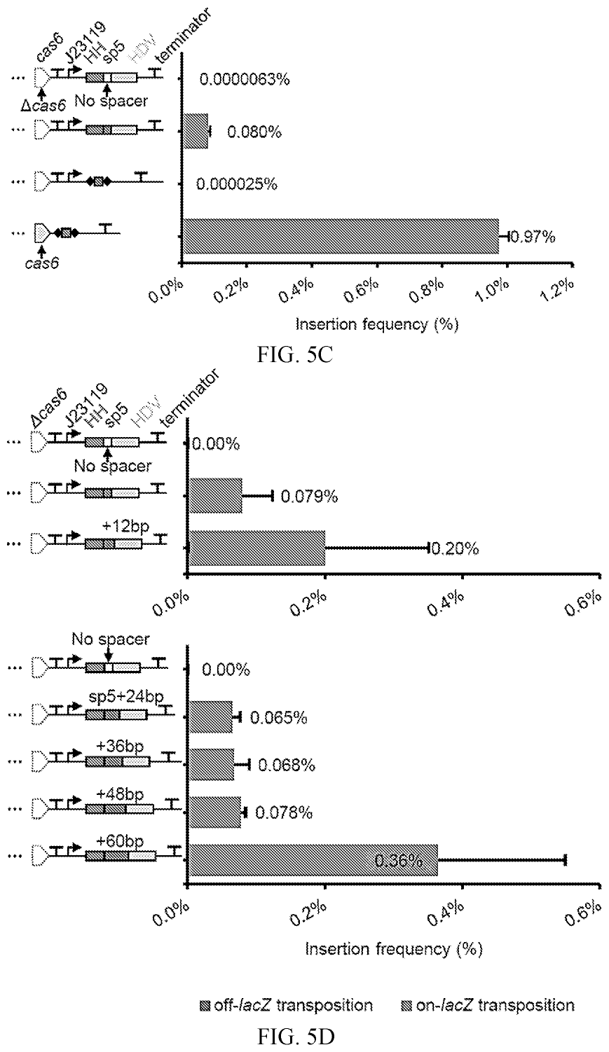

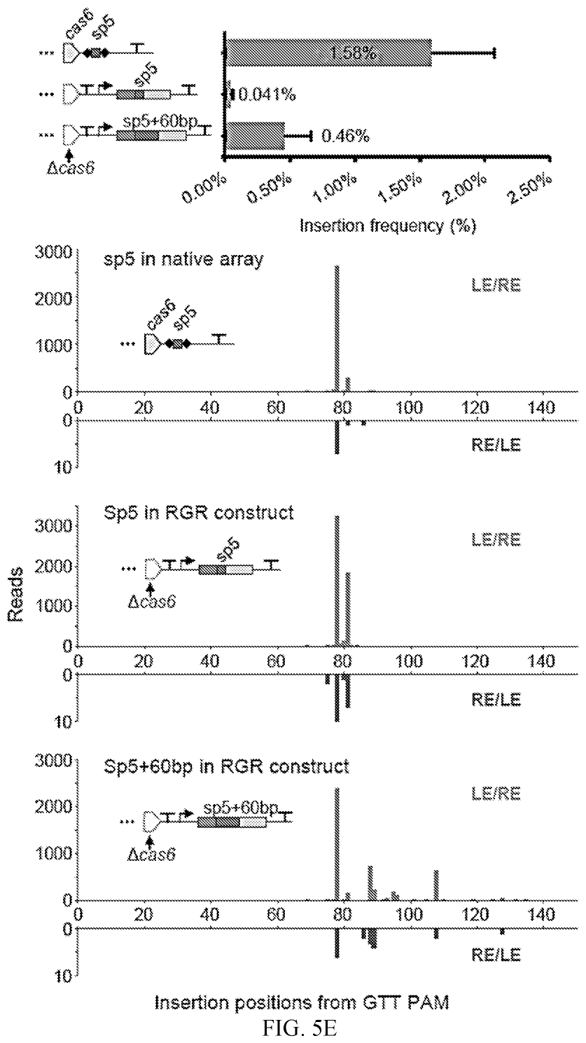

FIGS. 10A-C display transposition efficiency by spacer and the effect of mismatches. Specifically, FIG. 10A shows eight different spacers tested for transposition efficiency: four targeting the lacZ top strand, and four targeting the lacZ bottom strand. Right: All spacers are 35 bp with GTT PAM. Bottom: protospacer positions on lacZ to approximate scale. In all mate-out assays, n=3. The off-lacZ bar is not visible because it is less than one percent of the total. FIG. 10B displays the insertion distributions of mini-McCAST guided by different spacers. The location and orientation of the spacers are illustrated at the top. The insertion distribution of each spacer is estimated by mapping NGS reads onto the target F plasmid. The reads showing mini-McCAST inserted with the left end adjacent to the target (LE/RE) are plotted in red on the bar chart; the reads showing opposite insertion orientation (RE/LE) are plotted in blue on the inverse bar chart. Note: y-axes are not on the same scale. Note that the insertion profile data for spacers 1 and 5 are the same data as in FIGS. 2B and 5E, respectively, for comparison. FIG. 10C displays the frequency of transposition with lacZ spacer 1 variants with mismatches. Positions of mismatches of each spacer variant are marked red at the bottom diagram. (sc: scrambled lacZ spacer 1 as negative control). Data are shown as mean+SD, n=3.

### Example 3

This example demonstrates that type I-D McCAST element shows the PAM preference found with canonical I-D elements.

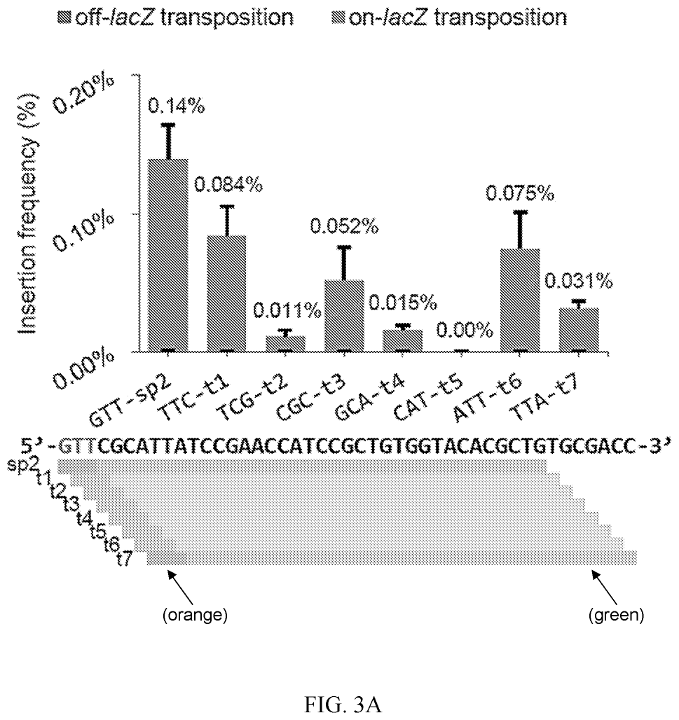

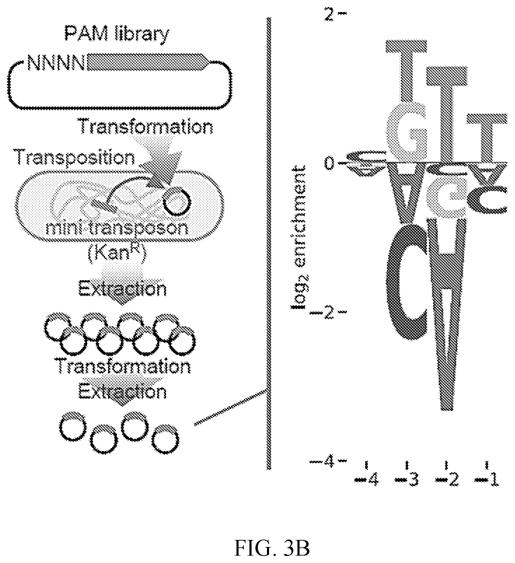

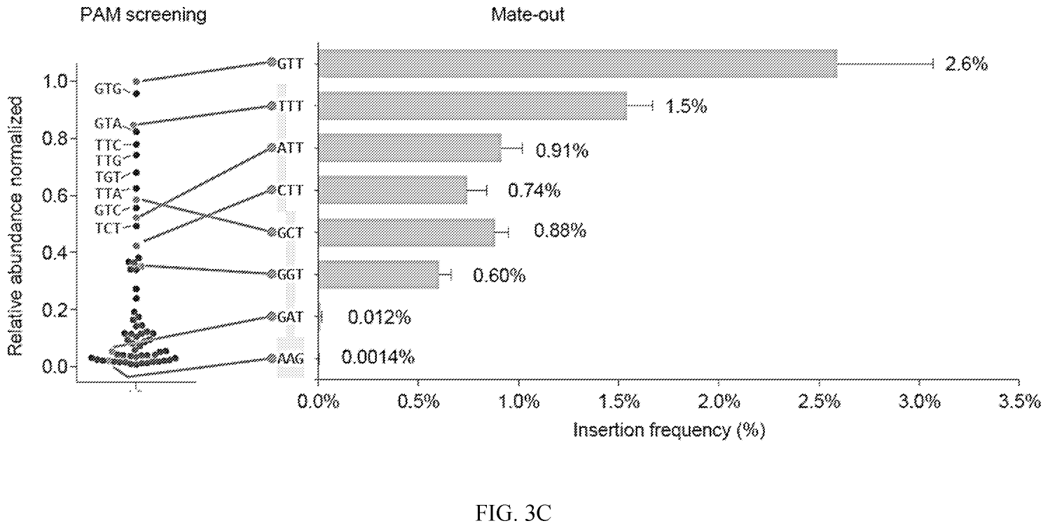

Canonical I-F1 systems strongly prefer a CC PAM, while diverse type I-F3 CAST show high levels of PAM promiscuity and in one case, an element (Tn7479) lacks any PAM requirement. To get more information on the sequence requirements of the type I-D CAST system, this example monitored transposition frequency and targeting when crRNA was tiled downstream relative to lacZ spacer 2. The tiling spacer experiment showed that most spacers with non-GTN PAM on their targets allow low, but detectable levels of guide RNA-directed transposition (FIG. 3A). A PAM screen was conducted to investigate the PAM requirement of the type I-D McCAST system. A PAM library was made on a target plasmid with the most efficient protospacer we identified and used as a transposition target in vivo (FIG. 10A). Plasmids in the library with preferred PAMs should be over-represented as targets in a population of cells capable of McCAST transposition, and anti-PAMs should be underrepresented following deep sequencing of the population.

PAM enrichment was measured by comparing the sequencing results of the PAM library before and after the screen. The type I-D McCAST showed no clear nucleotide preference at −4 position, while there was a clear G/T, T, T bias across the −3 through −1 positions (FIG. 3B). There were clear sequences that were also biased against, suggesting anti-PAMs exist in the type I-D CAST system (FIG. 3B). By plotting the normalized relative abundances of PAM sequences after screening as a swarm plot, the PAM requirement of McCAST aligns with the general GTN PAM of type I-D CRISPR systems, but in addition to GTN, TTN were also among the top performing PAMs (FIG. 3C). In contrast, the NAN PAMs were all disfavored by McCAST; considering the type I-D CRISPR repeat also has an A at its −2 position, it can serve as an anti-PAM signal to reduce self-targeting. Direct testing of selected PAMs in the mate-out transposition assay confirmed the PAM screening results. Although this example revealed an unusual preference toward TTN PAM and showed that some other PAMs can support a modest level of transposition (FIGS. 3B-C), McCAST does not have PAM promiscuity as observed in many type I-F3 CAST elements.

FIGS. 3A-C display the PAM preference of McCAST. FIG. 3A demonstrates that spacers are tiled along lacZ gene in a 1-bp increment from lacZ spacer 2. The transposition efficiency of each spacer is determined with the mate-out assay. The 3 nucleotide PAM of each spacer is labeled. Data is shown as mean+SD. The off-lacZ transposition rates are too low to be visible in the bar chart. The position of each tiled spacer is illustrated below; green bars indicate protospacers, and orange bars indicate PAM. FIG. 3B displays the PAM screening process, illustrated on the left. The enrichment of PAM is determined with deep sequencing the library before and after selection, log 2 scale enrichment of nucleotides at each position is shown on the right. FIG. 3C displays the relative abundance of PAMs normalized by the most abundant PAM are plotted on a swarm plot on the left. The PAMs with different nucleotides at the −4 position showed no clear preference at the position. To confirm the PAM screening results, eight different F plasmids carrying a lacZ fragment with different PAMs were constructed and tested for transposition efficiency with the mate-out assay with results indicated in the bar graph. Data are shown as mean+SD. The off-target rates are not measured in this experiment.

### Example 4

This example demonstrates that extended spacers are functional for type I-D McCAST transposition.

The CRISPR surveillance complexes of Class I CRISPR systems comprise multiple proteins and a crRNA; oligomerization of Cas7 family proteins on the RNA scaffold forms the backbone, while other proteins cap the ends. In many type I CRISPR-Cascades, Cas11 (small subunit) forms part of the complex on the guide RNA along with the Cas7 filament, similar to type III CRISPR-Cascades. In type I-A, I-E, the small subunit is encoded in a separate gene; while in type I-B, I-C, I-D, the small subunit is encoded within the large subunit gene (Cas8/Cas10) (FIG. 11A). Previous work on type I-F3 CAST Tn6677 found that shortening or extending a spacer greatly reduces the activity of RNA-guided transposition.

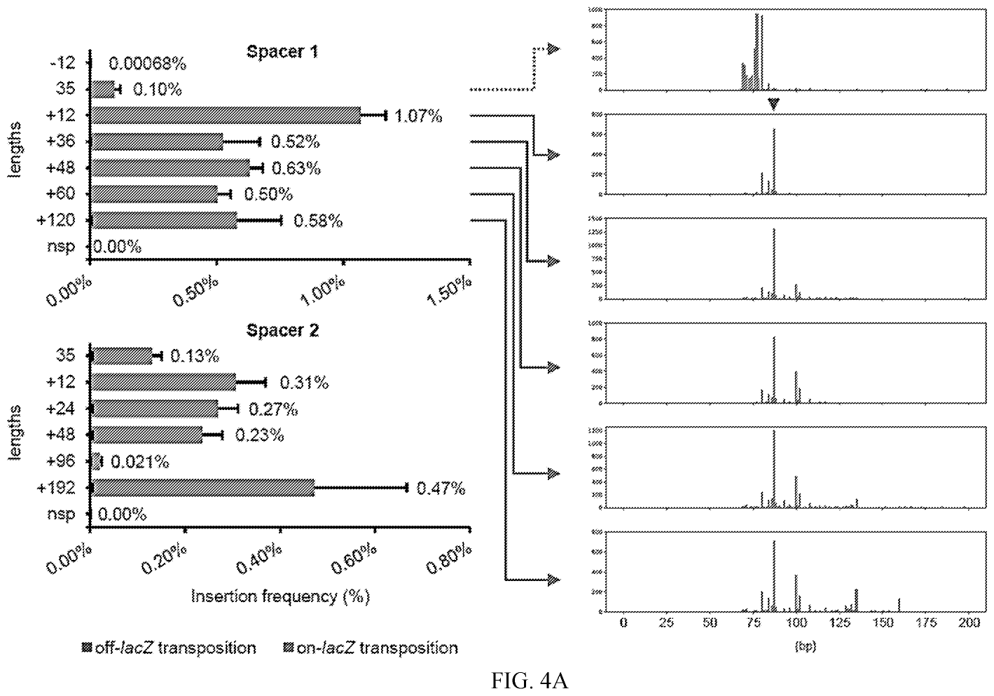

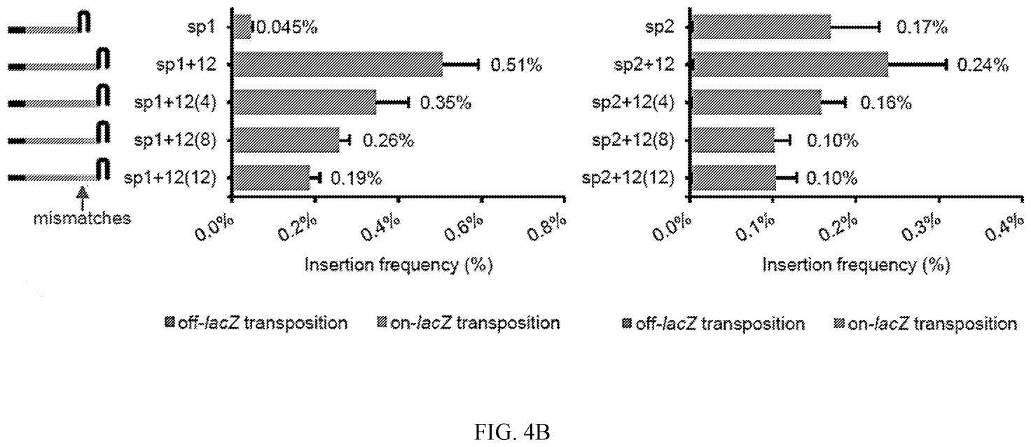

This example tested for changes in functionality with changes in guide RNA length in the type I-D McCAST system. While shortening the spacer by 12 bp greatly diminished transposition, extended spacers were functional and generally showed a higher frequency of transposition (FIGS. 4A-B, FIG. 12). Mapping insertions using NGS revealed that with the extended spacers, the resulting transposition events shift further downstream. While a portion of the transposition events could be shifted to increasing distances from the PAM with longer spacers, a prominent hotspot of insertions was fixed at ˜75 bp from the PAM. Longer guides revealed additional hotspots at ˜100 and ˜135 bp with this spacer. One possible explanation is that the crRNA of type I-D CRISPR-Cas in naturally occurring systems may be a mixed population with different lengths. A heterogeneous mix of type I-D CRISPR crRNAs was found when transcripts from a native host were examined with high-throughput transcriptome analysis; the less abundant transcripts differ in length by about 6 nt intervals, suggesting the trimming and natural variation in the number of Cas7. Recent structural studies on purified type I-D Cascade also observed the heterogeneity of the length of Cas7 filament. The pre-crRNA in type I and type III CRISPR-Cas systems is processed from the transcript by a Cas6 family endonuclease (Cas5 for type I-C) into functional guide RNAs. In some CRISPR-Cas families (type I-C, I-E, I-F), the nuclease remains part of the Cascade complexes. However, in other CRISPR-Cas subtypes, the Cas6 endonuclease dissociates (type III, I-A, I-B), and the crRNA is further processed at the 3′-end by an unresolved factor such as a host nuclease(s), usually resulting in a heterogeneous population of crRNAs.

Previous work showed the importance of the Cas11 subunit encoded within the Cas10 gene. This example overexpressed the Cas7 and Cas11 proteins under the hypothesis that more of these proteins could be needed with extended spacer to coat the longer guide RNAs, but overexpression of these components modestly reduced the frequency of transposition and did not alter the distribution of insertions (FIGS. 11A-B).

Extended spacers were also tested for their mismatch tolerance at the PAM distal extension. For CRISPR-Cas that were shown to be able to accommodate extended spacers, the type I-E CRISPR-Cas from E. coli was found to be susceptible to mismatches at the extension; on the contrary, the type I-F CRISPR-Cas from A. actinomycetemcomitans D7S-1 was found to be functional as long as its Cascade can form R-loop longer than 32 bp starting from 5′-end of spacer. An intermediate phenotype was found when the type I-D McCAST system was examined (FIG. 4B, FIG. 12). The results differed slightly depending on the initial activity of the spacer chosen. Generally, increasing the length of the mismatched segment at the distal end modestly reduced transposition. Nonetheless, extended spacers are functional in McCAST.

FIGS. 4A-B display the impact of extended spacers on McCAST transposition and the resulting insertion distributions. FIG. 4A demonstrates the lacZ spacers 1 and 2 with altered lengths tested for transposition using the mate-out assay. The results are shown on the left as mean+SD. Spacers with native length are labeled with 35 (bp), and spacers with altered lengths are labeled with the number of nucleotides increased or decreased. nsp: the negative control without the spacer. Transposition events with selected spacers were mapped with deep sequencing as indicated. Note that the insertion profile data in the top panel in part A is the same data as in FIG. 2B for comparison (indicated with a dotted line). FIG. 4B demonstrates the effect of having mismatches on the distal part of extended spacers was tested with the mate-out assay and shown as mean+SD. The numbers of mismatches are labeled in parentheses. In all mate-out assays, n=3. The off-lacZ bar is not visible because it is less than one percent of the total.

FIGS. 11A-B display the effect of expressing additional Cas11d, Cas7d with extended spacers. FIG. 11 A displays that the McCAST cas11d start codon is identified by aligning the protein sequence with the Cas10d of Synechocystis sp. PCC 6803, whose cas11d start codon had been confirmed. FIG. 11B (left) demonstrates that two extended spacers were tested with or without expressing additional Cas11d and Cas7d, lacZ spacer 1 with 60 bp extension and lacZ spacer 2 with 96 bp extension. Expressing additional Cas1 Id and Cas7d fails to improve the transposition rate. In all mate-out assays, n=3. The off-lacZ bar is not visible because it is less than one percent of the total. Additional Cas11-Cas7 expression vector was made by subcloning the Cascade operon as shown on top. FIG. 11B (right) displays the insertion distribution found with lacZ spacer 1 with a 60 bp extension is almost identical with and without expressing additional Cas1 Id and Cas7d.

FIG. 12 displays the effect of having mismatches at the extended region of the spacers. Transposition efficiency with different lacZ targeting spacers and their variants tested with the mate-out assay. +12: the spacer is extended by 12 bp; (12) the 12 bp from the end of spacer is mismatched to the protospacer. In all mate-out assays, n=3. The off-lacZ bar is not visible because it is less than one percent of the total.

### Example 5

This example demonstrates that the type I-D McCAST system can be engineered for simplified guide RNA maturation and independence from Cas6.

A previous type I-D Cascade from Synechocystis sp. PCC6803 when expressed in E. coli showed Cas6 co-purified with full-length crRNA with the same stoichiometry as the complex. To analyze Cas6d dispensability for guide RNA-directed transposition in the I-D McCAST system, this example removed the downstream repeat normally required for Cas6d processing and binding at the 3′ end of the guide RNA complex. Removing the 3′ repeat reduced transposition, but on-target transposition events were still detected, implying Cas6d activity was not essential (FIG. 5A). Constructs with an extended spacer did not compensate for the loss of Cas6d processing (FIG. 5A). To directly test if Cas6d was an essential component of the effector complex involved in type I-D McCAST transposition, cas6 gene was deleted and a ribozyme-catalyzed system was used for guide RNA production. In this synthetic construct, a constitutive heterologous J23119 promoter drives the expression of a guide RNA that functions as a self-processed ribozyme guide ribozyme (RGR) construct (FIG. 5B). The RGR construct was initially developed to overcome the limitations of gRNA processing in non-native settings. Processing occurs via hammerhead and the hepatitis delta virus (HDV) ribozymes self-cleaving at the 5′ and 3′ of crRNA, respectively, thereby removing the need for native Cas6 processing activity. This construct allowed for directly testing Cas6d dependence and provided another mechanism of altering the length of guide RNAs. Guide RNAs produced in the systems were functional for guide RNA-directed transposition in the absence of Cas6d, while the same spacer cannot guide transposition without Cas6d in the context of a normal array (FIG. 5C). Transposition rates varied with guide RNA length with the auto-processed RGR construct (FIG. 5D), but the same general profile of insertions was found with the Cas6-processed and auto-processed RGR constructs (FIG. 5E). Guides engineered to be +60 in length showed the same hotspot as the 35 base spacer, although a portion of the insertions extended from the hotspot with the extended construct (FIG. 5E).

FIGS. 5A-E provide an analysis of the requirement of Cas6d for RNA-guided transposition. FIG. 5A displays the transposition efficiency of different array variants determined by the mate-out, data shown as mean+SD. Array structures are illustrated on the left. From top to bottom are lacZ spacer 1 with additional 12 nucleotides flanked by native repeats, the same spacer with downstream repeat removed, lacZ spacer 1 increased by 120 nucleotides with downstream repeat removed, PaqCI entry sites flanked by native repeats without target spacer. FIG. 5B displays a schematic of the crRNA processing by Cas6d and crRNA processing by ribozyme-guide-ribozyme (RGR) construct. HH: hammerhead ribozyme, HDV: Hepatitis delta virus ribozyme. FIG. 5C displays the frequency of transposition found with lacZ spacer 5 in the RGR construct or normal array or normal array construct without cas6. RGR construct without spacer is used as the control of lacZ spacer 5 in RGR construct. FIG. 5D displays that the transposition efficiencies of lacZ spacer 5 with different lengths in RGR construct were determined with the mate-out assay and shown as mean+SD. RGR construct were tested at various lengths with PaqCI entry sites is used as no spacer control. The experiments are in two panels because they are done at two different times. FIG. 5E compares the transposition rates of lacZ spacer 5 in a normal array construct with lacZ spacer 5 (and its extended version increased by 60 nucleotides) in the RGR construct. The frequency of transposition was determined with the mate-out assay and shown in mean+SD. The resulting insertions were mapped with deep sequencing. The scales of reads of two insertion orientations are different. In all mate-out assays, n=3. The off-lacZ bar is not visible because it is less than one percent of the total.

### Example 6

This example demonstrates that the type I-D McCAST element moves by cut-and-paste transposition.

The entire tRNA-targeting branch where the McCAST and PmcCAST elements belong have TnsA and TnsB as a single polypeptide (FIG. 1). In spite of having the TnsA nuclease domain, in a previous study, PmcCAST was found to form cointegrates at roughly the rate as found with TnsA-free type V-K CAST, suggesting its TnsAB fusion protein lacked the expected TnsA nuclease activity.

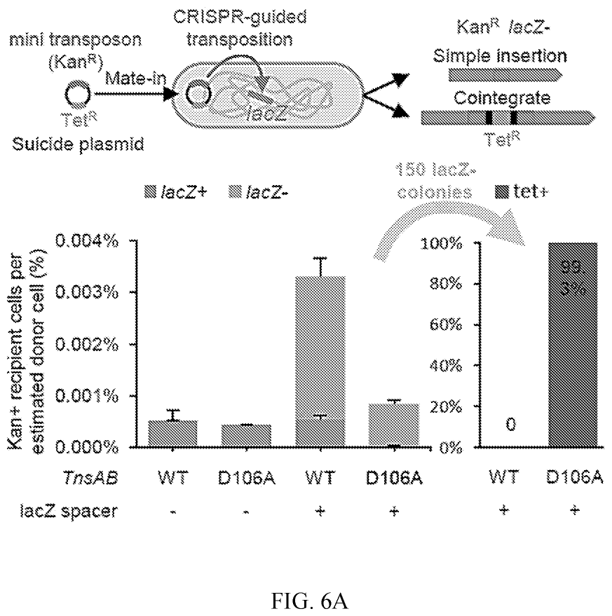

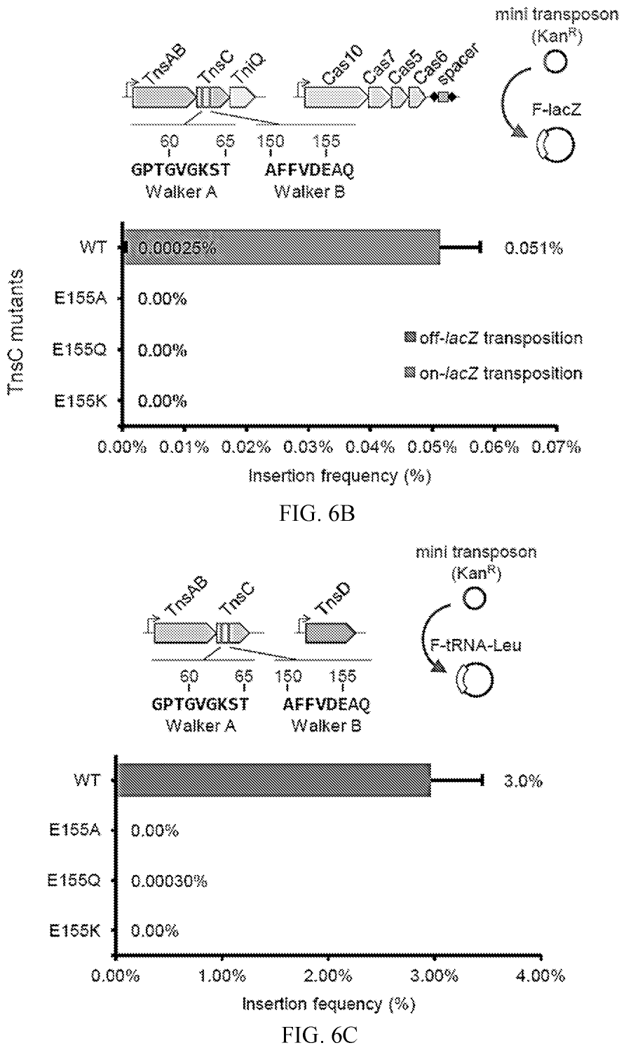

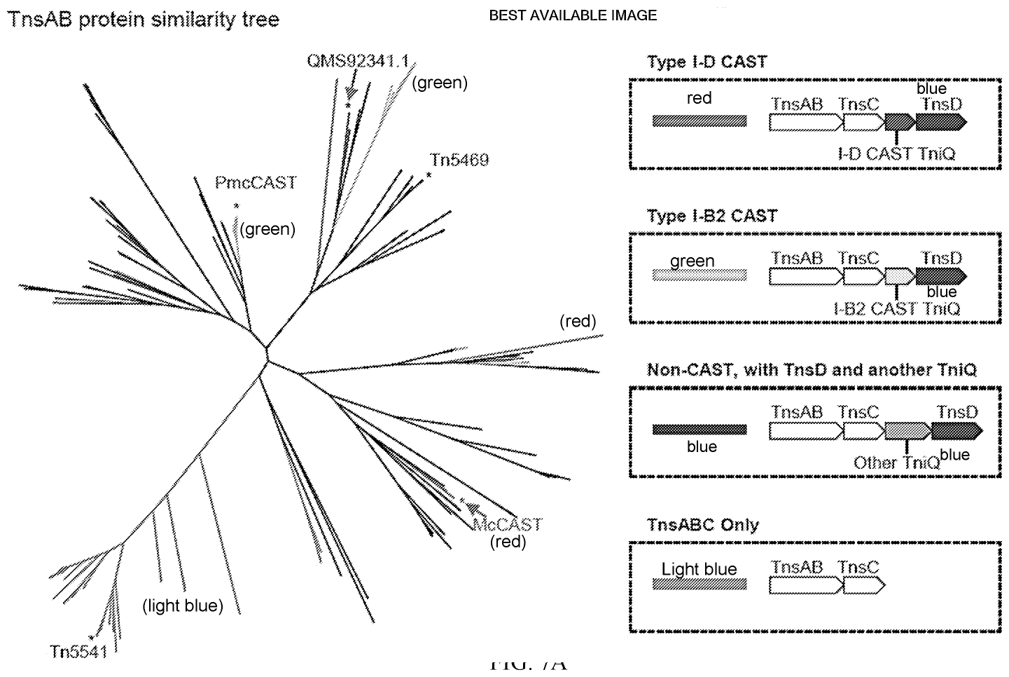

To investigate the TnsA activity of McCAST, which is a relative of PmcCAST (TnsAB a.a. identity 54%), a transposition assay was developed to measure the cointegrate rate with McCAST transposition. The assay utilizes a mate-in strategy to deliver a conditional donor plasmid into host cells where plasmid replication is not maintained. The use of the mate-in assay with a conditional plasmid helps guard against potential toxicity that could result from integrating a second origin of DNA replication into the chromosome, something that could favor confounding RecA-mediated cointegrate resolution. As in the mate-out transposition assays described above, transposition of the mini-McCAST element was directed to protospacers in the lacZ locus to estimate successful guide RNA-targeted transposition on agar selection plates containing X-Gal. Targeted transposition required the lacZ spacer in the assay (FIG. 6A). The incidence of cointegrates could be monitored phenotypically because the conditional vector backbone used in this assay encoded resistance to tetracycline (TetR). On-target transposition events in the lacZ gene (white colonies on X-gal) were screened for TetR which indicated that none of the transposition events with the type I-D CAST system were cointegrates (0/150) (FIG. 6A). As a control for the assay, an active site mutant predicted to inactive the nuclease activity in the TnsA active site and result in cointegration was also tested. In the TnsAB(D106A) mutant, nearly all the transposition events were stable cointegrates (149/150), indicating these could be readily detected in the assay when present (FIG. 6A). These experiments indicate that the TnsAB fusion found in the McCAST element has a functional TnsA nuclease activity catalyzing transposition via a cut-and-paste mechanism in the heterologous E. coli host.

The core machinery of Tn7-like transposons is composed of a transposase TnsB and an AAA+ ATPase regulator protein, TnsC. TnsC forms the functional connection between the transposase and the target site selection proteins, playing roles in transposase activation and target immunity. Structural studies showed that in the type V-K ShCAST system TnsC directly interacts with target selection protein TniQ, and its ATPase activity is essential for transposition. In prototypic Tn7, ATPase activity of TnsC is also required for targeted transposition. While mutating the TnsC Walker B motif in type I-F3 CAST and V-K CAST systems abolishes transposition, inactivating Tn7 TnsC ATPase by mutating its Walker B motif resulted in unregulated random transposition. Different Walker B mutations of McCAST TnsC were tested and it was found that the predicted loss of ATPase activity impairs both RNA-guided and TnsD-guided transposition pathways (FIG. 6B).

FIGS. 6A-C display the characteristics of McCAST. FIG. 6A displays the examination of TnsA activity with the mate-in assay. The experimental procedure is illustrated. The mini-McCAST element was encoded on a conditionally replicative plasmid that is transferred into recipient cells expressing RNA-guided transposition machinery via conjugation from donor cells. Four kinds of recipient cells are used, those with and without lacZ spacer 1 and having TnsAB(D106A) mutation or TnsAB wild type (WT). The amount of recipient cell colonies carrying mini-McCAST marker per donor cell (%) are shown on the left bar chart. LacZ+ colonies are indicated in blue, while LacZ− colonies are indicated in white. White colonies were only found when the lacZ spacer was expressed, supporting RNA-guided transposition into the lacZ gene. Cointegrate formation was judged by testing for the plasmid backbone marker allowing TetR (n=150), supporting cut-and-paste transposition in the wild-type configuration and coinetegrate formation in the TnsA(D106A) nuclease mutant (Right).

FIG. 6B displays different Walker B motif mutants tested for their ability to support transposition. The Walker motif sequences and their positions are indicated. The key glutamate residue (E155) required for ATP hydrolysis is marked in red. All E155 mutants inactivate both transposition pathways, suggesting that ATP hydrolysis is required for transpositions. In all mate-out assays, n=3. The off-lacZ bar is not visible because it is less than one percent of the total.

FIG. 6C displays TnsAB binding sites arrangement on the ends of tRNA-targeting transposons with TnsAB fusion in cyanobacteria. Only examples where both ends and the expected target site duplication could be identified are included. Identical sequences were removed. The TnsAB binding site arrangement is unique among known Tn7-like elements. The asterisk marks the McCAST.

### Example 7

This example demonstrates that the TGT/ACA end sequence is not universally conserved in Tn7-like transposons.

The ends of Tn7-like family transposons have multiple TnsB binding sites set in an asymmetric arrangement that allows control over insertion orientation. The distribution of TnsAB binding sites differed from most other Tn7-like element families; TnsAB binding sites are found in both orientations in the left end instead of a single orientation as found in other elements (FIG. 6C). Most Tn7-like transposons experimentally investigated thus far are bounded by 5′-TGT/ACA-3′. McCAST, however, is bounded by 5′-TAC/GTA-3′. Changing the McCAST ends to 5′-TGT/ACA-3′ had a modest effect on transposition frequency and showed no increase in off-site targeting outside lacZ (FIG. 13A), consistent with the idea that the end sequence requirement is not as strict as originally assumed.

The transposon ends of Tn7-like transposons with tnsAB fusion and identifiable target-site-duplication in cyanobacteria with loosened criteria were searched and it was found that almost 20% of transposons do not have 5′-TGT/ACA-3′ ends (FIG. 13B). Although the mechanism behind the conserveness of 5′-TGT/ACA-3′ is yet to be understood, the 5′-TGT/ACA-3′ is not universally conserved.

As shown in FIG. 13A, changing the terminal sequence of McCAST from 5′-TAC/GTA-3′ to 5′-TGT/ACA-3′ does not abolish the transposition. Two spacers were tested, lacZ spacer 1 (left), lacZ spacer 1 with 12 bp extension (right). FIG. 13B displays the frequencies of predicted transposon terminal sequences (Left/Right) of Tn7-like transposons with tnsAB fusion in cyanobacteria. Only those with the target site duplication are counted. In all mate-out assays, n=3. The off-lacZ bar is not visible because it is less than one percent of the total.

### Example 8

This example demonstrates the extensive targeting flexibility and evidence of convergent evolution with Tn7-like elements in cyanobacteria.

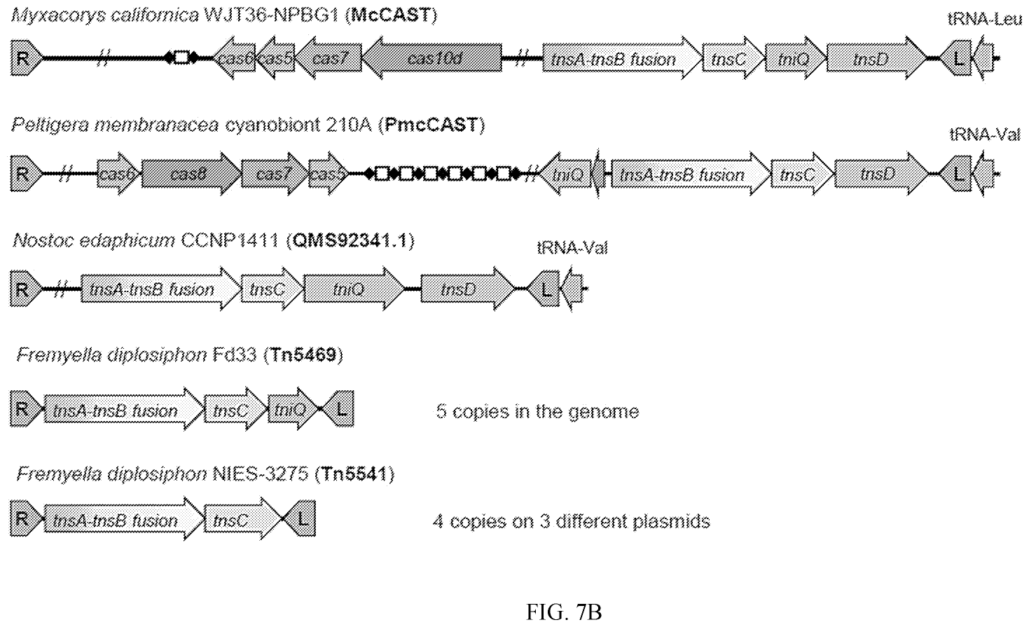

The Cas-coopting TniQ of type I-B2 and I-D CAST systems form their own phylogenetic clades indicating a single origin for each of these groups (FIG. 1, panel C). However, within the TnsAB elements examined in this disclosure, it was found that type I-B2 and type I-D CAST do not cluster into distinct branches but scatter across branches of the TnsAB similarity tree (FIG. 7A). This indicates that there are horizontal exchanges of type I-B2 and I-D systems between elements. Within the TnsAB Tn7-like elements, Tn5469, an element previously identified as a spontaneous insertion mutagen in cyanobacteria, was also found. The Tn7-like Tn5469 element has no cargo and only one TniQ and was identified in a screen for spontaneous inactivation of a gene, consistent with the idea that the element inserts without targeting an attachment site of a specific DNA sequence. The even simpler Tn5541 Tn7-like element with a TnsAB fusion and TnsC but lacking TniQ and cargo also likely lacks dual targeting pathway choice. The Tn5541 branch of elements has an extra extension at the C-terminal of its TnsC and only appears to be on plasmids in the sequenced representatives suggesting a novel type of targeting preference may be found with these elements found in cyanobacteria.

A bioinformatic analysis indicated that convergent evolution is a repeating theme with Tn7-like elements. Convergent evolution has repeatedly selected diverse tRNA genes as targets by guide RNAs or as fixed sites directly recognized by a DNA binding domain (FIG. 1, panel C). This example also found examples of convergent evolution with Tn7-like elements acquiring targeting pathways directed at attachment sites where insertion inactivates genes responsible for natural transformation (genetic competency) (FIGS. 8A-C).

Applying the same analysis used to discover the type I-D CAST systems, this example identified multiple cases where the type I-B1 CASTs use the guide RNA system to target an attachment site in the chromosome. Multiple examples where candidate competence (com) genes are targets for guide RNA-directed transposition were discovered. Multiple examples were found where the final guide RNA encoded in the CRISPR array also targets the comM gene and in another case where the comEC gene is targeted (FIG. 8A-B). As part of this analysis, a different kind of mobile element was also identified, a tyrosine recombinase-based integrating element that also targets the comM gene (FIG. 8C).

FIGS. 7A-B display the diversity and evolutionary flexibility of Tn7-like transposons with TnsAB fusion in cyanobacteria. FIG. 7A displays the unrooted TnsAB protein similarity tree of Tn7-like transposons in cyanobacteria. The branches that belong to transposons with a putative tRNA-targeting TnsD and an additional TniQ protein are colored based on the putative functions of the second TniQ. The legend indicates coopting type I-B2 Cas: green, coopting type I-D Cas: red, others: blue. The transposons without TniQ are colored light blue. Other CAST systems are marked with an asterisk and labeled. FIG. 7B displays the gene arrangements of labeled transposons are illustrated.

FIGS. 8A-C display the convergent evolution observed in type I-B1 CASTs. FIG. 8A displays the TniQ protein similarity tree of Tn7-like transposons with a separate TnsA. TniQ proteins encoded in the same transposon are connected with curved lines. The type I-B1 CASTs are indicated in blue. The putative type I-B1 CRISPR coopting TniQ and glmS targeting TnsD are labeled. Some transposons with only type I-B1 CRISPR coopting TniQ are found to carry a chromosome attachment site targeting spacer (att-spacer) similar to results found with type I-F3 and type V-K CASTs. The att-spacers and protospacers of the transposons are illustrated, their positions on the tree are marked with numbers (*: the transposon is not on the tree because its tnsB is a pseudogene). All contained an ATG PAM as expected with type I-B CRISPR-Cas systems. Multiple transposons target comM, one targets comEC, and another targets an unknown hydrolase. FIG. 8B displays the comEC targeting transposon ends and protospacer sequence. The transposon is also predicted to have non-TGT/ACA ends. FIG. 8C displays a mobile genetic element with tyrosine recombinases also targets comM from the indicated GenBank accession.

The foregoing examples are intended to illustrate embodiments of the disclosure but are not intended to be limiting.

1. A system for use in DNA modification, the system comprising recombinantly produced or isolated type I-D CRISPR-associated transposon (CAST) proteins, wherein the system optionally does not include a Cas6 protein, the CAST proteins comprising:
i) a TnsC protein; a TnsD protein; a TniQ protein; a fusion protein comprising TnsA and TnsB proteins, a Cas5 protein, Cas7 protein, and a Cas10 protein; and
ii) a guide-RNA comprising a sequence targeted to a target within a DNA substrate.

2. The system of claim 1, wherein at least one of the TnsC protein, the TnsD protein, the TniQ protein, or the fusion protein, comprises an amino acid sequence that is at least 50% identical to a protein that is encoded by Myxacorys californica WJT36-NPBG1.

3. The system of claim 2, wherein:
a) the system comprises a ribozyme component, wherein the ribozyme component is capable of processing a precursor of the guide RNA, and wherein the ribozyme component is present on the precursor of the guide RNA, or the ribozyme is provided as a separate polynucleotide; and/or
b) the guide RNA comprises at least one protein binding site that is not a Cas6 binding site, or comprises a polynucleotide binding site, or a combination thereof.

4. The system of claim 3, wherein the system does not include the Cas6 protein.

5. The system of claim 3, comprising the ribozyme component.

6. The system of claim 3, wherein guide RNA comprises the at least one protein binding site that is not the Cas6 binding site.

7. The system of claim 3, wherein the guide RNA comprises the polynucleotide binding site.

8. The system of claim 7, wherein the polynucleotide binding site is an RNA primer binding site.

9. The system of claim 8, further comprising a reverse transcriptase.

10. The system of claim 1, wherein at least one of the CAST proteins is modified to include a nuclear localization signal or an amino acid linker sequence or a combination thereof.

11. A method comprising introducing cells of a system of claim 1, wherein the system modifies a DNA substrate in the cells that is targeted by the guide RNA.

12. The method of claim 11, wherein the system comprises the ribozyme component or an expression vector encoding the ribozyme, wherein the ribozyme processes a precursor of the guide RNA to form the guide RNA.

13. The method of claim 11, wherein the system does not include the Cas6 protein.

14. The method of claim 11, wherein the guide RNA comprises at least one protein binding site that is not a Cas6 protein binding site, or a polynucleotide binding site, or a combination thereof.

15. The method of claim 14, wherein at least one of the CAST proteins is modified to include a nuclear localization signal, an amino acid linker sequence, or a combination thereof.

16. The method of claim 15, wherein the system further comprises a DNA insertion template that is inserted into a location of the DNA substrate, said location being targeted by the guide RNA.

17. The method of claim 16, wherein the modification of the DNA substrate does not comprise a double stranded break of the DNA substrate.

18. A cell comprising the system of claim 1, wherein the cell is not Myxacorys californica WJT36-NPBG1.

19. A ribonucleoprotein comprising recombinantly produced or isolated type I-D CRISPR-associated transposon (CAST) proteins, wherein the ribonucleoprotein system optionally does not include a Cas6 protein, the CAST proteins comprising a TnsC protein, a TnsD protein, a TniQ protein, a fusion protein comprising TnsA and TnsB proteins, a Cas5 protein, Cas7 protein, a Cas10 protein, and a guide-RNA comprising a sequence targeted to a target within a DNA substrate

20. The ribonucleoprotein of claim 19, wherein ribonucleoprotein does not include a Cas6 protein.

21. The ribonucleoprotein of claim 20, wherein the guide RNA comprises at least one protein binding site that is not a Cas6 binding site, or a polynucleotide binding site, or a ribozyme component, or a combination thereof.

22. One or more expression vectors that encode:
i) a TnsC protein;
ii) a TnsD protein;
iii) a TniQ protein; and
iv) a fusion protein comprising TnsA and TnsB proteins, wherein at least one of the TnsC protein, the TnsD protein, the TniQ protein, or the fusion protein, comprises an amino acid sequence that is at least 50% identical to a protein that is encoded by Myxacorys californica WJT36-NPBG1.

23. The one or more expression vectors of claim 22, wherein the one or more expression vectors also encode a guide RNA or a precursor of the guide RNA.
